(19) United States
(12) Patent Application Publication
(10) Pub. No.: US 2022/0050117 A1
(43) Pub. Date: Feb. 17, 2022

(54) GLX-DERIVED MOLECULE DETECTION

(71) Applicant: GLX Analytix ApS, København N (DK)

(72) Inventors: Brian William Della Valle, København N (DK); Casper Hempel, Dyssegård (DK)

(21) Appl. No.: 17/274,741

(22) PCT Filed: Sep. 12, 2019
(86) PCT No.: PCT/EP2019/074411

§ 371 Date: Mar. 9, 2021

(30) Foreign Application Priority Data
Sep. 12, 2018 (EP) 18194086.7

(51) Int. Cl. G01N 33/68

(52) U.S. Cl. CPC G01N 33/6893; G01N 2333/70596; G01N 2333/70585; G01N 2400/40

(57) ABSTRACT

The present invention relates to biomarkers associated with immune-mediated inflammatory disease (IMID), particular GLX molecules, and even more particular GLX-related glycosaminglycans (GAGs) and GLX-related proteoglycans (PGs).

The present invention relates to the diagnosis and/or prognosis of inflammatory diseases. More particularly it concerns measurement of biomarkers for the pre-diction of the risk of developing and/or suffering from inflammatory diseases in a subject. Moreover, the invention relates to a medicament or substance for the treatment or prevention of inflammatory diseases in an individual, the surveil-lance of the treatment success and the choice of treatment.

### BACKGROUND OF THE INVENTION

An immune-mediated inflammatory disease (IMID) is any of a group of conditions or diseases which are characterized by common inflammatory pathways leading to inflammation, and which may result from, or be triggered by, a dysregulation of the normal immune response. All IMIDs can cause end organ damage, and are associated with increased morbidity and/or mortality.

Inflammation is an important and growing area of biomedical research and health care because inflammation mediates and is the primary driver of many medical disorders such as autoimmune diseases (e.g. ankylosing spondylitis, psoriasis, systemic lupus erythematosus psoriatic arthritis, Bechet's disease, rheumatoid arthritis, inflammatory bowel disease (IBD), Type I Diabetes (T1DM) and allergy), as well as many cardiovascular, neuromuscular (e.g. Amyotrophic Lateral Sclerosis (ALS)), neurodegenerative (e.g. Parkinson's Disease (PD)), Multiple System Atrophy (MSA), Alzheimer's Disease (AD), psychiatric (e.g. Major Depression, Bipolar, Schizophrenia, Anxiety Disorder), Neurodevelopmental disease and infectious diseases.

IMID is characterized by immune dysregulation, and one underlying manifestation of this immune dysregulation is the inappropriate activation of inflammatory cytokines, such as IL-12, IL-6 or TNF alpha often combined with a reduced production of anti-inflammatory cytokines, whose actions lead to pathological consequences.

While there is evidence for disease associations, progress is being limited by research being pursued in a disease-specific manner, according to clinical presentation, and often being focused on latter stages of disease. Consequently, the early mechanisms surrounding loss of immune tolerance or early inflammatory instigators have been overlooked.

Patent document WO2009068685 extensively discloses the general principles detection, diagnosis, a high-throughput device, and use of such a device for said purpose.

Despite the advent of proteomics for assessing plasma biomarkers, treatment strategies are challenged by the lack of pathognomonic biomarkers predicting disease severity, early signs of new disease flares, and response-to-treatment.

The prevalence of IMID in Western society is estimated at 10%. Currently, there are no straightforward methods to identify common disease characteristics and thus there continues to be an unmet need for biomarkers usable in predicting the risk of developing an IMID, which potentially could lead to organ dysfunction, failure and chronic disease. Additionally, there is a need for further determining the specific type of inflammatory disease, and on this background initiate targeted and effective treatment courses.

### SUMMARY OF THE INVENTION

In a first aspect of the invention, one or more of these objects are solved by an ex vivo method of diagnosing and/or prognosticating at least one immune-mediated inflammatory disease (IMID) in a subject, said method comprising the steps of
a. performing in vitro measurement of the level of one or more biomarkers selected from the group consisting of glycosaminoglycans (GAGs) and proteoglycans (PGs) of the glycocalyx in a first biological sample from the subject;
b. optionally, performing in vitro measurement of the level of the one or more biomarkers in a second or further biological samples from the subject obtained at a later time point than said first biological sample.
c. comparing the level of said one or more biomarkers to the level of one or more corresponding references; and
d. using the measured value to evaluate the state of the subject.

In a further embodiment, the glycosaminoglycan (GAG) is Keratan Sulfate and/or the proteoglycan (PG) is selected from BiGlycan, Glypican-1, Glypican-4, Syndecan-3 and/or CD44.

In a further embodiment the level of two or more biomarkers is measured.

In a further embodiment, the state of the subject evaluated in step d. is determined as at risk of developing and/or having a given IMID, if said level of two or more biomarkers are above and/or below the corresponding one or more reference levels, and determined as not at risk of developing or having IMID, if said one or more levels are equal to one or more corresponding reference levels.

In yet another embodiment the level of the one or more biomarkers is significantly greater or lower than the corresponding reference value with a P-value of at least <0.05.

The glycocalyx is a layer composed of proteoglycans, glycoproteins and glycolipids that covers the cell membranes of most epithelial animal cells. Generally, the constituents of the glycocalyx are involved in signal transduction, cell-cell recognition, communication, intercellular adhesion (leukocytes and thrombocytes) and maintain vessel-wall homeostasis.

In blood vessels, the endothelial glycocalyx is located on the apical surface facing the lumen. The glycocalyx, which is located on the apical surface of endothelial cells, is composed of a negatively charged network of proteoglycans, glycoproteins, and glycolipids. When vessels are stained with cationic dyes, transmission electron microscopy shows an irregularly shaped and diverse layer extending into the lumen of a blood vessel. It is present throughout a diverse range of microvascular beds (including capillaries) and macrovessels (arteries and veins).

The glycocalyx barrier reinforces the vessel and organ against diseases. Another main function of the glycocalyx within the vascular endothelium is that it shields the vascular walls from direct exposure to blood flow, while serving as a vascular permeability barrier. Its protective functions are universal throughout the vascular system. Another protective function throughout the cardiovascular system is its ability to affect the filtration of interstitial fluid from capillaries into the interstitial space.

Because the glycocalyx is so prominent throughout the cardiovascular system, disruption to this structure has detrimental effects that can cause disease. Initial dysfunction of the glycocalyx leads to internal fluid imbalance, and potentially edema, leukocyte invasion, disrupting the barrier function and potentially the endothelial cell itself leading to cascading deleterious effects of inflammation.

Shedding of the glycocalyx can be triggered by inflammatory stimuli, such as tumor necrosis factor-alpha. Whatever the stimulus is, however, shedding of the glycocalyx leads to a cascade of dysregulation, dysfunction and permissibility to damage of the vessel and the organ it is serving.

In one embodiment of the invention, the biological sample is a blood sample, such as whole blood, plasma, serum or cerebrospinal fluid.

In a further embodiment of the invention at least one additional biomarker is selected from one or more of the group consisting of chondroitin sulfate (CS), heparan sulfate (HS), hyaluronic acid (HA) and dermatan sulfate (DS), keratan sulfate (KS) and/or selected from one or more of the group consisting of syndecans, glypicans, and biglycans. The term “one additional biomarker” means one biomarker in addition to another biomarker, or one biomarker in addition to two or more biomarkers.

In yet another embodiment, the syndecan is selected from one or more of the group consisting of syndecan-1, syndecan-2, syndecan-3 and syndecan-4 and/or the glypican is selected from one or more of the group consisting of, glypican-1, glypican-2, glypican-3, glypican-4, glypican-5 and glypican-6 and or, the proteoglycan (PG) is selected from one or more of the group consisting of biglycan, CD44, perlecan, mimecan, decorin, hyalectans (aggrecan, versican, neurocan, brevican), betaglycan, lumincan, keratocan.

In a particular embodiment of the invention, the biomarkers are two or more biomarkers selected from the group consisting of BiGlycan, CD44, Keratan Sulfate, GPC-1, GPC-4, Hyaluronic Acid, Chondroitin Sulfate, Perlecan, Heparan Sulfate, Syndecan1, Syndecan2, Syndecan3, and Syndecan4.

It is contemplated that the immune-mediated inflammatory disease diagnosed or prognosticated according to the invention may be or develop into an autoimmune disease, a neuroinflammatory disease, neurodegenerative disease, psychiatric disease an organ system disease, or more specifically be or develop into one of the following diseases of table 1 and which could be diagnosed or prognosticated by the method of the invention. The individual diseases could be diagnosed or prognosticated either alone or simultaneously in a single biological sample:

TABLE 1
List of inflammatory diseases

Autoimmune
Achalasia
Addison's disease
Adult Still's disease
Agammaglobulinemia
Alopecia areata
Amyloidosis
Ankylosing spondylitis
Anti-GBM/Anti-TBM nephritis
Antiphospholipid syndrome
Autoimmune angioedema
Autoimmune dysautonomia
Autoimmune encephalomyelitis
Autoimmune hepatitis
Autoimmune inner ear disease (AIED)
Autoimmune myocarditis
Autoimmune oophoritis
Autoimmune orchitis
Autoimmune pancreatitis
Autoimmune retinopathy
Autoimmune urticaria
Axonal & neuronal neuropathy (AMAN)
Bab disease
Behcet's disease
Benign mucosal pemphigoid
Bullous pemphigoid
Castleman disease (CD)
Celiac disease
Chagas disease
Chronic inflammatory demyelinating
polyneuropathy (CIDP)
Retinitis
Chronic recurrent multifocal osteomyeli-
tis (CRMO)
Churg-Strauss Syndrome (CSS) or Eo-
sinophilic Granulomatosis (EGPA)
Cicatricial pemphigoid
Cogan's syndrome
Cold agglutinin disease
Congenital heart block
Coxsackie myocarditis
CREST syndrome
Crohn's disease
Dermatitis herpetiformis
Dermatomyositis
Devic's disease (neuromyelitis optica)
Discoid lupus
Dressler's syndrome
Endometriosis
Eosinophilic esophagitis (EoE)
Eosinophilic fasciitis
Erythema nodosum
Neurodevelopmental
Essential mixed cryoglobulinemia
Evans syndrome
Fibromyalgia
Fibrosing alveolitis
Giant cell arteritis (temporal arteritis)
Giant cell myocarditis
Glomerulonephritis
Goodpasture's syndrome
Granulomatosis with Polyangiitis
Graves' disease
Guillain-Barre syndrome
Hashimoto's thyroiditis
Hemolytic anemia
Henoch-Schonlein purpura (HSP)
Herpes gestationis or pemphigoid ges-
tationis (PG)
Hidradenitis Suppurativa (HS) (Acne In-
versa)
Hypogammalglobulinemia
IgA Nephropathy
IgG4-related sclerosing disease
Immune thrombocytopenic purpura
(ITP)
Inclusion body myositis (IBM)
Interstitial cystitis (IC)
Juvenile arthritis
Juvenile diabetes (Type 1 diabetes)
Juvenile myositis (JM)
Kawasaki disease
Lambert-Eaton syndrome
Leukocytoclastic vasculitis
Lichen planus
Lichen sclerosus
Ligneous conjunctivitis
Linear IgA disease (LAD)
Lupus
Lyme disease chronic
Meniere's disease
Microscopic polyangiitis (MPA)
Mixed connective tissue disease
(MCTD)
Mooren's ulcer
Mucha-Habermann disease
Multifocal Motor Neuropathy (MMN) or
MMNCB
Wegener's granulomatosis (or Granulo-
matosis with Polyangiitis (GPA))
Myasthenia gravis
Myositis
Narcolepsy
Neonatal Lupus
Neuromyelitis optica
Neutropenia
Ocular cicatricial pemphigoid
Optic neuritis
Palindromic rheumatism (PR)
PANDAS
Paraneoplastic cerebellar degeneration
(PCD)
Paroxysmal nocturnal hemoglobinuria
(PNH)
Parry Romberg syndrome
Pars planitis (peripheral uveitis)
Parsonage-Turner syndrome
Pemphigus
Peripheral neuropathy
Perivenous encephalomyelitis
Pernicious anemia (PA)
POEMS syndrome
Polyarteritis nodosa
Polyglandular syndromes type I, II, III
Polymyalgia rheumatica
Polymyositis
Postmyocardial infarction syndrome
Postpericardiotomy syndrome
Primary biliary cirrhosis
Primary sclerosing cholangitis
Progesterone dermatitis
Psoriasis
Psoriatic arthritis
Pure red cell aplasia (PRCA)
Pyoderma gangrenosum
Raynaud's phenomenon
Reactive Arthritis
Reflex sympathetic dystrophy
Relapsing polychondritis
Restless legs syndrome (RLS)
Retroperitoneal fibrosis
Rheumatic fever
Rheumatoid arthritis
Sarcoidosis
Schmidt syndrome
Scleritis
Scleroderma
Sjogren's syndrome
Sperm & testicular autoimmunity
Stiff person syndrome (SPS)
Subacute bacterial endocarditis (SBE)
Susac's syndrome
Sympathetic ophthalmia (SO)
Takayasu's arteritis
Temporal arteritis/Giant cell arteritis
Thrombocytopenic purpura (TTP)
Tolosa-Hunt syndrome (THS)
Transverse myelitis
Type 1 diabetes
Ulcerative colitis (UC)
Undifferentiated connective tissue dis-
ease (UCTD)
Uveitis
Vasculitis
Vitiligo
Vogt-Koyanagi-Harada Disease
NeuroInflammatory/Neurodegenerative
Stroke
Alzheimer's Disease
Parkinson's Disease
Menigitis
Traumatic Brain Injury
Chronic Traumatic Encephalopathy
Epilepsy
Demyelinating Disease
Motor Neuron Disaese
Amyotrophic Lateral Sclerosis
Mild Cognitive Impairment
Dementia
Encephalitis
Other Inflammatory
Chronic Kidney Disease
COPD
Diabetes
Type II Diabetes
Chronic Liver Disease
Cirrohosis
Pericarditis
Pancreatitis
Myocarditis
Arthritis
Osteoarthritis
Bronchiolitis
Vasculitis
Dermatitis
Encephalitis
Lymphangitis
Hepatitis
Vasculitis
Atherosclerosis
Glomerulonephritis
Nephritis
Glomerulosclerosis
Hepatitis
Steatohepatitis
Endocarditis
Myocarditis
Chorioamnionitis
Thyroiditis
Organ System diseases
(Brain)
Disorder (e.g. Au-
tism)
Huntington's Disease
Creutzfeldt-Jakob Disease
Prion Disease
(Cardiovascular)
Heart Disease
Myocardial Infarction
Coronary Heart Disease
Connective Tissue Disease
(Liver Disease)
Fatty Liver
Liver Disease
Non-alcoholic liver disease
Alcoholic liver disease
(Kidney Disease)
Nephropathy
(Lung Disease)
COPD
Pulmonary Fibrosis
Sarcoidosis
Tuberous Sclerosis
(Muscluar Disease)
Myopathy
Neuromusclar Disease
Pain
(Pregnancy)
PreEclampsia
Placental Infarction
Fibrinoid necrosis
Pyschiatric Disorders
Mental Disorder
Depression
Psychosis
Schizoprenia
Anxiety Disorder
Depressive Disorder
Major Depression
Bipolar disorder
Psychotic disorders

The amount of biological sample tested is more than or equal to 0.01, μl such as 0.02 μl, 0.05 μl, 0.1 μl, 0.5 μl, 1 μl, 2 μl, 5 μl or 10 μl. Larger volumes may be used depending on the assay.

In one embodiment of the ex vivo method according to the invention, step a) is performing in vitro measurement of the level of two, three, four, five, ten, twenty or more biomarkers selected. The measurement of the level of two or more biomarkers can be carried out simultaneously or consecutively, such as multiplexing of two or more biomarkers.

In another aspect of the invention the objects are solved by providing a substance for use in a method of treating condition in an individual known to have at least one or more biomarkers selected from the group consisting of glycosaminoglycans (GAGs) and proteoglycans (PGs) of the glycocalyx, wherein the biomarkers are at level above the corresponding one or more reference levels, wherein the substance and condition are selected from:

| Drug | IMID |
|---|---|
| Biologics | Autoimmune Disease |
| Non-steroidal anti-inflammatory drugs | |
| Glucocorticoids | |
| Disease-modifying antirheumatic drugs | |
| Biosimilars | |
| Vasodilators | Stroke |
| Statins | |
| Anticonvulsants | |
| ACE Inhibitors | |
| galantamine | Alzheimer's Disease |
| donepezil | |
| rivastigmine | |
| memantine | |
| citalopram | |
| mirtazapine | |
| sertraline | |
| bupropion | |
| duloxetine | |
| imipramine | |
| GLP-1 receptor agonists | |
| GIP receptor agonists | |
| SGLT-2 inhibitors | |
| Testosterone replacement | |
| memantine | |
| namzari | |
| Aducanumab | |
| Solanezumab | |
| Insulin | |
| verubecestat | |
| AADvac1 | |
| CSP-1103 | |
| intepiridine | |
| Levodopa | Parkinson's Disease |
| Carbidopa | |
| Carbidopa-levodopa | |
| Dopamine agonists | |
| MAO B inhibitors | |
| Catechol O-methyltransferase (COMT) | |
| inhibitors | |
| Anticholinergics | |
| Amantadine | |
| GLP-1 agonist | |
| GIP agonist | |
| SGLT-2 inhibitor | |

In one embodiment the GAG is Keratan Sulfate and/or the PG is selected from the list of BiGlycan, Glypican-1, Glypican-4, Syndecan-3 and CD44.

Furthermore, the objects are solved by providing a substance for use in a method of treating condition in an individual known to have at least one or more biomarkers selected from the group consisting of glycosaminoglycans (GAGs) and proteoglycans (PGs) of the glycocalyx, wherein the substance and condition are selected from:

| Substance: | Condition (IMID): |
|---|---|
| Immunosuppressant Drugs | Lupus |
| Cyclophosphamide | |
| Azathioprine | |
| Hydroxychloroquine | |
| Methotrexate | |
| Ibuprofen | |
| Naproxen | |
| Sulindac | |
| misoprostol | |
| Prednisolone | |
| methylprednisolone | |
| butesonide | |
| chloroquine | |
| dapsone | |
| chlorambucil | |
| mycophenolate mofetil | |
| Belimumab | |
| Rituximab | |
| dehydroepiandrosterone | |
| Immunosuppressant Drugs | Rheumatoid Arthritis |
| Cyclophosphamide | |
| Azathioprine | |
| Belimumab | |
| Hydroxychloroquine | |
| Rituximab | |
| Ibuprofen | |
| Etanercept | |
| methotrexate | |
| sulfasalazine | |
| leflunomide | |
| Infliximab | |
| prednisolone | |
| methylprednisolone | |
| butesonide | |
| Immunosuppressant Drugs | Inflammatory Bowel Disease |
| Mesalazine | |
| prednisolone | |
| methylprednisolone | |
| butesonide | |
| Azathioprine | |
| methotrexate | |
| cyclosporin | |
| tacrolimus | |
| adalimumab | |
| Infliximab | |
| certolizumab pegol | |
| Ornidazole | |
| Metronidazole | |
| clarithromycin | |
| rifaximin ciprofloxacin | |
| infliximab | Celiac Disease |
| infliximab-abda | |
| infliximab-dyyb | |
| balsalazide | Crohn's Disease |
| Mesalamine | |
| olsalazine | |
| Sulfasalazine | |
| Tacrolimus | |
| prednisolone | |
| methylprednisolone | |
| Butesonide | |
| Azathioprine | |
| methotrexate | |
| Cyclosporin | |
| Golimumab | |
| adalimumab-adbm | |
| Adalimumab | |
| Infliximab | |
| infliximab-abda | |
| infliximab-dyyb | |
| vedolizumab | |
| Immunosuppressant Drugs | |
| balsalazide | Ulcerative Colitis |
| Mesalamine | |
| olsalazine | |
| Sulfasalazine | |
| prednisolone | |
| methylprednisolone | |
| Butesonide | |
| Azathioprine | |
| methotrexate | |
| cyclosporin | |
| golimumab | |
| adalimumab-adbm | |
| adalimumab | |
| infliximab | |
| infliximab-abda | |
| infliximab-dyyb | |
| vedolizumab | |

Immunosuppressant Drugs

| | |
|---|---|
| Calcipotriene | Psoriasis |
| Acitretin | |
| Methoxsalen | |
| trioxsalen | |
| adalimumab | |
| Etanercept | |
| infliximab | |
| secukinumab | |
| ixekizumab | |
| apremilast | |
| methotrexate | |
| cyclosporin | |

Immunosuppressant Drugs

| | |
|---|---|
| atorvastatin | Stroke |
| fluvastatin | |
| lovastatin | |
| pitavastatin | |
| pravastatin | |
| rosuvastatin | |
| simvastatin | |
| Coumadin | |
| Jantoven | |
| Marfarin | |
| clopidogrel | |
| Tissue plasminogen activator | |
| angiotensin-converting enzyme inhibitors | |
| beta-blockers | |
| calcium channel blockers | |
| GLP-1 agonist | |
| GIP agonist | |
| SGLT-2 inhibitor | |
| galantamine | Alzheimer's Disease |
| donepezil | |
| rivastigmine | |
| memantine | |
| citalopram | |
| mirtazapine | |
| sertraline | |
| bupropion | |
| duloxetine | |
| imipramine | |
| GLP-1 receptor agonists | |
| GIP receptor agonists | |
| SGLT-2 inhibitors | |
| Testosterone replacement | |
| memantine | |
| namzaric | |
| Aducanumab | |
| Solanezumab | |
| Insulin | |
| verubecestat | |
| AADvac1 | |
| CSP-1103 | |
| intepiridine | |

and/or selected from

| | |
|---|---|
| INN | Common brand names |

Psychiatric Disease
Anxiety Disorders

| | |
|---|---|
| Alprazolam | Xanax |
| Bromazepam | Lexotanil |
| Chlordiazepoxide | Librium |
| Clobazam | Frisium |
| Clonazepam | Klonopin |
| Clorazepate | Tranxene |
| Diazepam | Valium |
| Lorazepam | Ativan, Temesta |
| Oxazepam | Serax |
| Tofisopam | Emandaxin, Grandaxin |

Non-Benzodiazepine Anxiolytics

| | |
|---|---|
| Buspirone | BuSpar, Spitomin |
| Hydroxyzine | Atarax, Vistaril |
| Meprobamate | Equanil, Miltown |
| Gabapentin | Neurontin, Gabaran |
| Pregabalin | Lyrica |

Antidepressants

| | |
|---|---|
| Citalopram | Celexa, Cipramil |
| Clomipramine | Anafranil |
| Doxepin | Doxepin, Sinequan |
| Escitalopram | Cipralex, Lexapro |
| Fluoxetine | Prozac, Sarafem |
| Fluvoxamine | Fevarin, Luvox |
| Imipramine | Tofranil |
| Mirtazapine | Avanza, Remeron, Zispin |
| Paroxetine | Paxil, Pexeva, Seroxat |
| Sertraline | Lustral, Zoloft |
| Trazodone | Azona, Deprax, Desyrel, Oleptro, Trittico, |
| | Thombran |
| 5-HTP | |
| Tryptophan | |

Autism

| | |
|---|---|
| Aripiprazole | Abilify |
| Risperidone | Risperdal |

Bipolar Disorder
Mood Stabilizers

| | |
|---|---|
| Carbamazepine | Carbatrol, Carnevix, Epitol, Equetro, Tegretol, |
| | Tegretol XR, Teril |
| Gabapentin | Neurontin |
| Lamotrigine | Lamictal |
| Levetiracetam | Keppra |
| Lithium salts | Camcolit, Eskalith, Lithobid, Sedalit |
| Oxcarbazepine | Trileptal |
| Topiramate | Topamax |
| Sodium valproate | Convulex, Depakene, Depakine Enteric, Orfiril, |
| [note 1] | Stavzor |
| Divalproex | Depakote, Epival, Ergenyl Chrono |
| sodium [note 2] | |
| Sodium valproate | Depakine Chrono, Depakine Chronosphere, |
| and | |
| valproic acid in | Epilim Chrono, Epilim Chronosphere |
| 2.3:1 ratio | |

Atypical Antipsychotics

| | |
|---|---|
| Aripiprazole | Abilify |
| Asenapine | Saphris, Sycrest |
| Olanzapine | Zyprexa |
| Quetiapine | Seroquel |
| Risperidone | Risperdal |
| Ziprasidone | Geodon, Zeldox |

Depressive Disorders

| | |
|---|---|
| Amisulpride | Amazeo, Amipride, Amival, Deniban, Solian, |
| | Soltus, Sulpitac, Sulprix |
| Amitriptyline | Elavil, Endep, Tryptanol, Tryptomer |
| Agomelatine | Valdoxan, Melitor, Thymanax |
| Bupropion | Aplenzin, Wellbutrin |
| Citalopram | Celexa, Cipramil |
| Clomipramine | Anafranil |
| Desipramine | Norpramin, Pertofrane |
| Desvenlafaxine | Pristiq |
| Doxepin | Aponal, Adapine, Deptran, Prudoxin, Silenor, |
| | Sinquan, Sinequan, Zonalon |
| Duloxetine | Cymbalta |
| Escitalopram | Cipralex, Lexapro |
| Fluoxetine | Prozac, Sarafem |
| Fluvoxamine | Luvox, Faverin |
| Imipramine | Antideprin, Tofranil |
| Lamotrigine | Lamictal |
| Levomilnacipran | Fetzima |
| Mirtazapine | Remeron, Avanza |
| Moclobemide | Aurorix, Manerix |
| Nortriptyline | Aventyl, Pamelor |
| Paroxetine | Paxil, Pexeva, Seroxat |
| Phenelzine | Nardil |
| Protriptyline | Vivactil |
| Reboxetine | Edronax, Norebox, Prolift, Solvex, Vestra |
| Rubidium | Rubinorm |
| chloride | |
| Selegiline | Emsam |
| Sertraline | Zoloft, Lustral |
| Tianeptine | Stablon, Coaxil, Tatinol |
| Tranylcypromine | Parnate |
| Trazodone | Azona, Deprax, Desyrel, Oleptro, Trittico, |
| | Thombran |
| Venlafaxine | Effexor, Effexor XR |
| Vilazodone | Viibryd |
| Vortioxetine | Trintellix |
| Chloral hydrate | Chloraldurat, Somnote |
| Clomethiazole | Distraneurin, Heminevrin |
| Glutethimide | Doriden |
| Niaprazine | Nopron |
| Sodium oxybate | Alcover, Xyrem |
| Tizanidine | Sirdalud, Zanaflex |
| Amitriptyline | Elavil, Endep, Laroxyl, Lentizol, Saroten, |
| | Sarotex, Tryptizol, Tryptomer |
| Doxepin | Doxepin, Silenor |
| Mianserin | Bolvidon, Depnon, Lerivon, Tolvon |
| Mirtazapine | Avanza, Remeron, Zispin |
| Trazodone | Azona, Deprax, Desyrel, Oleptro, Trittico, |
| | Thombran |
| Trimipramine | Rhotrimine, Stangyl, Surmontil |

Psychotic Disorders

| | |
|---|---|
| Chlorprothixene | Truxal |
| Levomepromazine | Levium, Levomepromazine Neuraxph, Neurocil |
| Perazine | Perazin Neuraxph, Taxilan |
| Promethazine | Atosil, Closin, Promethazin Neuraxph, |
| | Proneurin, Prothazin |
| Prothipendyl | Dominal |
| Sulpiride | Dogmatil, Dogmatyl, Sulpirid |
| Thioridazine | Mellaril, Thioridazin Neuraxph |
| Zuclopenthixol | Cisordinol, Clopixol |
| Perphenazine | Trilafon |
| Benperidol | Benperidol Neuraxph, Glianimon |
| Bromperidol | Impromen |
| Fluphenazine | decanoate |
| enanthate | Dapotum Injektion, Flunanthate, Moditen |
| | Enanthate Injection, Sinqualone Enantat |
| hydrochloride | Dapotum, Permitil, Prolixin, Lyogen, Moditen, |
| | Omca, Sediten, Selecten, Sevinol, Siqualone, |
| | Trancin |
| Fluspirilen | Fluspi, Fluspirilen Beta, Imap |
| Haloperidol | Haldol, Serenase |
| Pimozide | Orap |
| Amisulpride | Solian |
| Aripiprazole | Abilify |
| Asenapine | Saphris |
| Clozapine | Clozaril, Fazaclo, Leponex |
| Iloperidone | Fanapt |
| Lurasidone | Latuda |
| Melperone | Eunerpan, Melneurin |
| Olanzapine | Zyprexa, Zyprexa Relprevv |
| Paliperidone | Invega, Invega Sustenna |
| Quetiapine | Seroquel |
| Risperidone | Risperdal, Risperdal Consta |
| Ziprasidone | Geodon, Zeldox |
| Zotepine | Nipolept |
| Carbamazepine | Tegretol |
| Lamotrigine | Lamictal |

In a particular embodiment, the substance selected from the group composed of

| Drug | IMID |
|---|---|
| Biologics | Autoimmune |
| Non-steroidal anti-inflammatory drugs | Disease |
| Glucocorticoids | |
| Disease-modifying antirheumatic drugs | |
| Biosimilars | |
| atorvastatin | Stroke |
| fluvastatin | |
| lovastatin | |
| pitavastatin | |
| pravastatin | |
| rosuvastatin | |
| simvastatin | |
| coumadin | |
| jantoven | |
| marfarin | |
| clopidogrel | |
| tissue plasminogen activator | |
| angiotensin-converting enzyme inhibitors | |
| beta-blockers | |
| calcium channel blockers | |
| GLP-1 agonist | |
| GIP agonist | |
| SGLT-2 inhibitor | |
| galantamine | Alzheimer's |
| donepezil | Disease |
| rivastigmine | |
| memantine | |
| citalopram | |
| mirtazapine | |
| sertraline | |
| bupropion | |
| duloxetine | |
| imipramine | |
| GLP-1 receptor agonists | |
| GIP receptro agonists | |
| SGLT-2 inhibitors | |
| Testosterone replacement | |
| memantine | |
| namzari | |
| Aducanumab | |
| Solanezumab | |
| Insulin | |
| verubecestat | |
| AADvac1 | |
| CSP-1103 | |
| intepiridine | |
| Levodopa | Parkinson's |
| Carbidopa | Disease |
| Carbidopa-levodopa | |
| Dopamine agonists | |
| MAO B inhibitors | |
| Catechol O-methyltransferase (COMT) | |
| inhibitors | |
| Anticholinergics | |
| Amantadine | |
| GLP-1 agonist | |
| GIP agonist | |
| SGLT-2 inhibitor | |

for use in a method of treating a stroke, Parkinson's Disease (PD), Multiple System Atrophy (MSA), Amyotrophic Lateral Sclerosis (ALS), Type-I Diabetes Mellitus (T1DM) or Alzheimer's Disease (AD) in an individual known to have at least one or more biomarkers selected from the group consisting of glycosaminoglycans (GAGs) and proteoglycans (PGs) of the glycocalyx.

In one embodiment the GAG is Keratan Sulfate and/or the PG is selected from the list of BiGlycan, Glypican-1, Glypican-4, Syndecan-3 and CD44.

In another aspect of the invention, the above-stated objects are solved by providing a kit comprising
binding agents for at least two biomarkers selected from the group consisting of glycosaminoglycans (GAGs) and proteoglycans (PGs) of the glycocalyx; and
optionally instructions for using the binding agents in the evaluation of IMID.

In one embodiment the GAG is Keratan Sulfate and/or the PG is selected from the list of BiGlycan, Glypican-1, Glypican-4, Syndecan-3 and/or CD44.

Suitably, the kit may comprise
binding agents for Keratan Sulfate, BiGlycan, Glypican-1, Glypican-4, Syndecan-3 and/or CD44; and-instructions for using the binding agents in the evaluation of stroke, Parkinson's Disease (PD), Multiple System Atrophy (MSA), Amyotrophic Lateral Sclerosis (ALS), Type-I Diabetes Mellitus (T1DM) or Alzheimer's Disease (AD).

In yet another aspect, a microfluidic detection chip for the detection of an IMID infection in a patient, said chip comprising one or more separation channels (2) which are coated with one or more binding agents for the biomarkers selected from the group consisting of glycosaminoglycans (GAGs) and proteoglycans (PGs) of the glycocalyx.

In one embodiment the GAG is selected from Keratan Sulfate, Chondroitin Sulfate and Heparan Sulfate; and/or the proteoglycan (PG) is selected from BiGlycan, Glypican-1, Glypican-4, Syndecan-3 and/or CD44

In another embodiment the microfluidic detection chip for the detection of at least one IMID in a patient comprises one or more layers (3) in which a plurality of separation channels (2) are disposed in each layer; at least one sample inlet port (4) which is in fluid communication with the separation channels (2) and into which a biological sample is introduced; wherein one or more of the separation channels (2) have a plurality of three-dimensional (3D) separation zones (5); with an intermittent partition zone wherein the chip is an optical chip.

In one embodiment, the kit according to the invention further comprises said microfluidic chip wherein the one or more separation channels are coated with said binding agents specific for one or more biomarkers of GAGs and PGs.

### BRIEF DESCRIPTION OF FIGURES

The invention will be described in more detail below by means of non-limiting example of embodiments and with reference to the FIGS. 1-10.

FIG. 1 shows the results of a dot blotting analysis of glycocalyx-associated blood biomarkers for stroke. The biomarker tested are a) syndecan-3, b) chondroitin sulfate (CS), c) CD44, d) heparan sulfate (HS), e) syndecan-4, f) syndecan-1, g) glypican-1 and h) hyaluronic acid (HA).

FIG. 2 shows glycocalyx (GLX) components detected in the plasma of healthy individuals (HI) and longitudinally after ischemic stroke (ISS). Plasma from HI and patients day ≤3, day 7, and day 90 after ISS were immunoassayed for GLX markers.

FIG. 3 shows a correlational heatmap profile of GLX components detected in the plasma of healthy individuals and longitudinally after ISS.

FIG. 4 shows GLX components detected in the plasma of patients suffering from Parkinson's Disease (PD), Multiple System Atrophy (MSA) and Amyotrophic Lateral Sclerosis (ALS) and age matched healthy controls (HC).

FIG. 5 shows a correlational heatmap profile of GLX components detected in the plasma of patients suffering from Parkinson's Disease (PD), Multiple System Atrophy (MSA) and Amyotrophic Lateral Sclerosis (ALS) and age matched healthy controls (HC).

FIG. 6 shows GLX components detected in the plasma of patients suffering from Type-I Diabetes Mellitus (T1DM) in children (age 6-9) and in plasma of healthy sibling controls without T1DM.

FIG. 7 shows a correlational heatmap profile of GLX components detected in the plasma of patients suffering from Type-I Diabetes Mellitus (T1DM) in children (age 6-9) and in plasma of healthy sibling controls without T1DM.

FIG. 8 shows GLX components detected in the plasma of patients suffering from Alzheimer's Disease (AD) and age matched healthy controls (HC).

FIG. 9 shows a correlational heatmap profile of GLX components detected in the plasma of patients suffering from Alzheimer's Disease (AD) and age matched healthy controls (HC) as well as correlational heatmap profiles of GLX components after a 6-month treatment (Placebo vs. Treatment).

FIG. 10 shows CD44 detected in the plasma of patients suffering from Alzheimer's Disease (AD) after a 6-month treatment vs. receiving placebo.

### DETAILED DESCRIPTION

In the following, embodiments of the invention will be described in further detail. Each specific variation of the features can be applied to other embodiments of the invention unless specifically stated otherwise.

The biomarkers of the current invention are soluble components of the thick matrix lining of blood vessels termed the endothelial glycocalyx (GLX). This layer maintains vascular homeostasis and multiple disruptive stimuli leads to shedding of soluble and detectable components and thus loss of this layer. A decrease of GLX in the blood, can be due, in part, to inflammation-driven degeneration of the vessel or organ and/or ageing and/or gene repression. This translates into changes in plasma values and we have developed a very sensitive assay using only minute amounts of blood for detecting these changes. These biomarkers may predict risk of or diagnose diseases of the vasculature and brain e.g. Alzheimer's disease, generalized dementia, Parkinson's Disease, ALS, Depression, Schziophrenia, Traumatic Brain Injury, lupus, arthritis, inflammatory bowel disease, thyroiditis, Guillain-Barre syndrome, and vasculitis. There are a wide-range of diseases that may benefit from such an invention inclusive of many rare diseases of specific classes such as but not limited to autoimmune diseases, chronic inflammatory diseases, neurological disease, degenerative disease, psychiatric disease, and cardiovascular disease. The specific diseases are listed in table 2.

The GLX comprises a combination of different proteoglycans, glycolipids and/or glycosaminoglycans. The combinations of these molecules, as detected in biological fluids or samples, are different in different types of diseases, e.g. Alzheimer's vs Parkinson's Disease. In this manner, different diseases, present in different organ systems, will be able to predict disease course, predict attacks, and even diagnose disease in complement with other techniques such as MRI.

It is contemplated that GLX-cleaving enzymes are activated during immune invasion into diseased organs or alternatively, by auto-activated immune cells destined to cause disease. Indeed, GLX-removing enzymes are increased in the blood of a number of diseases.

It is further contemplated that a composite biomarker for GLX shedding would improve personalized approaches to disease monitoring and design therapies on a precision medicine principle. Thus, the identification of the entire structure of GLX shedding components in the blood, and the integration of these markers into a composite biomarker, a GLX profile or fingerprint or signature allows for the detection of disease, severity and a treatment response.

It is contemplated that a mechanism of action whereby an immune cell arrives at an endothelial cell, secretes enzymes to remove the GLX structure, transmigrates into a given organ and causes damage. Moreover, GLX shedding may be downregulated in the blood due to gene repression and organ degeneration.

Diseases that are inflammatory based would be top targets for the diagnostic, and treatments that are anti-inflammatory or disease-modifying in nature would be a primary use for testing treatment efficacy and separating treatment responders from non-responders.

Secondarily, very many diseases result in damage to an organ and inflammation follows. This is, for example the case with Stroke and epilepsy and Parkinson's Disease, though there are very many diseases for which secondary inflammation and even chronic inflammation play a part.

Therefore, this invention would also be valuable for diseases of this nature, to follow the damage caused, the expansion of the damage and a resolution of the disease, as would accompany a reduction in the biomarker. Additionally, this could be coupled to treatment efficacy on the underlying disease, since inflammation is a secondary marker of disease severity, and also opens value for separation of treatment responders and non-responders.

It is further contemplated that the biomarkers may be used in a method to distinguish between organ systems. This will be of value to determine where in the body the inflammation is coming from. For example, inflammation from a Lupus patient can be from the kidney or skin.

The assay for detection of the biomarkers is antibody-based, wherein the antibody binds to a specific epitope of the biomarker. A labelled secondary antibody is used to visualize the antibody-antigen complex for example via chemiluminescence detection system based on HRP enzymatic activity or fluorescence or near-infrared emission. As an alternative, the linking of lanthanides may be used.

The method for identification of the GLX-biomarkers may be immunostrips. Immunostrips are immunosensors where the recognition agent is an antibody that binds to the analyte with detection by reflectance or fluorescence spectrophotometry.

Detection systems suitable for detecting the biomarkers of the present invention includes but is not limited to immunoassays based on specific antibodies, lanthanides, lectins, GAG-binding molecules (table 2), Alcian Blue, Toluidine blue or dimethylmethylene blue.

TABLE 2
GAG-Binding Proteins
Fibroblast growth factors (FGF) - Important possibility

| | |
|---|---|
| Antithrombin | |
| Enzymes | GAG biosynthetic enzymes, thrombin and coagulation factors (proteases), |
| | complement proteins (esterases), extracellular superoxide dismutase, |
| | lipases |
| Enzyme inhibitors | antithrombin III, heparin cofactor II, secretory leukocyte proteinase |
| | inhibitor, C1-esterase inhibitor |
| Cell adhesion proteins | P-selectin, L-selectin, some integrins |
| Extracellular matrix proteins | laminin, fibronectin, collagens, thrombospondin, vitronectin, tenascin |
| Chemokines | platelet factor IV, γ- and β-interferons, interleukins, CXCL8, CXCL12, |
| | CCL2, CCL5 and CCL7 |
| Growth factors | fibroblast growth factors, hepatocyte growth factor, vascular endothelial |
| | growth factor, insulin-like growth factor-binding proteins, TGF-β-binding |
| | proteins |
| Morphogens | hedgehogs, TGF-β family members, wnts |
| Guidance factors | slits, ROBO receptors, neuropilins |
| Tyrosine-kinase growth factor | fibroblast growth factor receptors, vascular endothelium growth factor |
| receptors and coreceptors | receptor, RAGE, RPTPs |
| Lipid-binding proteins | apolipoproteins B, E, and A-V, lipoprotein lipase, hepatic lipase, annexins |
| Plaque proteins | prion proteins, amyloid proteins |
| Nuclear proteins | histones, transcription factors |
| Pathogen surface proteins | malaria circumsporozoite protein |
| Viral envelope proteins | herpes simplex virus, dengue virus, Zika virus, human immunodeficiency |
| | virus, hepatitis C virus, VCP |

Other methods for identification if the GLX-biomarkers may be mass spectrometry (MS) such as MALDI-MS, LC-MS, HPLC-MS or Liquid chromatography-electrospray ionization-tandem mass spectrometry.

The platform for detecting one or more biomarkers in one sample (so called multiplexing) may be in a chip-based device.

A kit will comprise a panel of all GLX markers deemed valuable as biomarkers for a specific disease. These will be detecting molecules (e.g. antibodies) that are chemically attached to a surface (e.g. a plate or a tube) which will specifically bind each individual GLX marker. This kit will take one biological sample and test for all GLX markers in one run, comparing to reference values. This is a multiplex assay that will provide a readout of each marker compared to reference and determine whether a given disease is active or, in the case of treatment response and/or autoimmune disease, in remission.

Chip-based technologies combining these elements where the device will send the signal generated through a printout, bluetooth, WIFi/Internet, and/or remote sensoring technology.

The chip system may be a microfluidics chip.

### Definitions

Prior to discussing the present invention in further details, the following terms and conventions will first be defined:

### Immune-Mediated Inflammatory Disease

Immune-mediated inflammatory disease (IMID) is any of a group of conditions or diseases which are characterized by common inflammatory pathways leading to inflammation, and which may result from, or be triggered by, a dysregulation of the normal immune response. All IMIDs can cause end organ damage, and are associated with increased morbidity and/or mortality. An IMID could be any disease from the list given in table 1.

### GLX Molecules

The “GLX” or “glycocalyx” is the carbohydrate-rich outer part of the cell surface of the majority of cells in the body, including the luminal endothelium. This layer is the first interaction between the blood and the vessel wall, both throughout the body and at the blood-brain barrier (BBB). As described in here, shedding of the GLX may be an early stage predictor of autoimmune and neurodegeneration, disease severity, and treatment efficacy. Examples of GLX molecules are Glycosaminoglycans (GAGs) and Proteoglycans (PGs). These may be brain derived. Thus, the term “GLX-related” is to be understood as molecules associated with (or has been associated with) the GLX structure. Phrased in another way, the “GLX-related” may be understood as molecules originating from the GLX structure.

### Glycosaminoglycans

Glycosaminoglycan (GAGs) or “mucopolysaccharides” are long unbranched polysaccharides consisting of a repeating disaccharide unit. Examples of Glycosaminoglycan (GAGs) forming part of the present invention are:
Keratan Sulfate: Keratan Sulfate (KS) is a β-1,3-linked poly-N-acetyllactosamine with sulfate residues on the 6 position of galatose and N-acetylglucosamine.
Chondroitin sulfate: Chondroitin sulfate (CS) is a sulfated glycosaminoglycan (GAG) composed of a chain of alternating sugars (N20 acetylgalactosamine and glucuronic acid). It is usually found attached to proteins as part of a proteoglycan.
Hyaluronic acid: Hyaluronic acid (HA) also called hyaluronan, is an anionic, nonsulfated GAG distributed widely throughout connective, epithelial, and neural tissues. It is unique among glycosaminoglycans in that it is non-sulfated, forms in the plasma membrane instead of the Golgi apparatus, and can be very large, with its molecular weight often reaching the millions. It is composed by dimers of D-glucoronic acid and N-acetylglucosamine.
Heparan sulfate: Heparan sulfate (HS) is a linear polysaccharide found in all animal tissues. It occurs as a proteoglycan (HSPG) in which two or three HS chains are attached in close proximity to cell surface or extracellular matrix proteins. It is sulfated and composed by dimer of glucuronic acid and N-acetylglucosamine.
Dermatan Sulfate (DS): DS is a linear polysaccharide formed by disaccharides containing GalNAc and IdoA with β linkages (1.4 or 1.3 respectively)

### Proteoglycans

Proteoglycans (PGs) are proteins that are heavily glycosylated. The basic proteoglycan unit consists of a “core protein” with one or more covalently attached glycosaminoglycan (GAG) chain(s). Examples of proteoglycans forming part of the present invention are:
Syndecans: Syndecans are single transmembrane domain proteoglycans that are thought to act as co-receptors, especially for G protein-coupled receptors. These core proteins carry heparan sulfate (HS) and chondroitin sulfate (CS) chains. The syndecan protein family has four members; Syndecan 1-4.
Glypicans: Glypicans (GPC or GP) constitute one of the two major families of heparan sulfate proteoglycans, with the other major family being syndecans. Six glypicans have been identified in mammals, and are referred to as GPC1-GPC6 or GP1-GP6.
Biglycan: Biglycan is a small leucine-rich repeat proteoglycan (SLRP) which is found in a variety of extracellular matrix tissues and often has GAG chains attached.
CD44-CD44 is a cell-surface PG involved in cell-cell interactions, cell adhesion and migration and acts as a receptor to hyaluronic acid and other ligands.
Perlecan—a modular HSPG that regulates various biological processes primarily because of its widespread distribution
Hyalectans (Aggrecan, versican, neurocan, brevican)—PGs with a tridomain structure involving hyaluronan binding domain, GAG side chain, and lectin bindgin domain.
Mimecan—also known as osteoglycin, a PG associated with KS GAGs and small leucine repeats.
Keratocan—a KS PG containing three chains of KS, a highly-sulfated linear polymer of N-acetyl-lactosamine covalently linked to asparagine residues via a mannose-containing oligosaccharide.
Lumincan—KS PG that interacts with Fas ligand and regulating sheddase activity.
Decorin—small leucine rich PG with functional activity in a wide variety of processes.
Betaglycan—a membrane bound PG with high transforming growth factor beta binding.

### Reference Level

In the context of the present invention, the term “reference level” relates to a standard in relation to a quantity, which other values or characteristics can be compared to. In one embodiment of the present invention, it is possible to determine a reference level by investigating the abundance, such as an elevated or lowered level in relation to the reference level, of one or more of the biomarkers according to the invention in biological or blood samples from healthy subjects. By applying different statistical means, such as multivariate analysis, one or more reference levels can be calculated. Based on these results, a cutoff may be obtained that shows the relationship between the level(s) detected and patients at risk. The cut-off can thereby be used to determine the amount of the one or more biomarkers, which corresponds to for instance an increased risk of PD.

A reference can consist of the biomarker level(s) of a single sample, or comprise an average value of biomarker level(s) from two or more samples. Alternatively two or more single samples can be considered as two or more references, respectively.

Furthermore, it is also possible to compare the biomarker levels of several biomarkers to their corresponding reference level to obtain a “profile” for the analyzed sample. Such a profile can comprise more than one dimension, such as elevated and decreased levels of several biomarkers and/or increased or decreased relationships between the markers.

### Risk Assessment

The present inventors have successfully developed a new method to predict the risk for developing a stroke, AD, ALS, PD, T1DM and/or MSA for a subject. The results presented in the examples show that the described biomarkers (alone or in combination) appear to be efficient biomarkers for determining whether a patient has an increased risk of developing stroke, AD, ALS, PD, T1DM and/or MSA.

To determine whether a patient has an increased risk of developing a stroke, AD, ALS, PD, T1DM and/or MSA or having an incident of acute inflammatory attack such as lupus attack, a cut-off must be established. This cut-off may be established by the laboratory, the physician or on a case-by-case basis for each patient. The cut-off level could be established using a number of methods, including: multivariate statistical tests (such as partial least squares discriminant analysis (PLS-DA), random forest, support vector machine, etc.), percentiles, mean plus or minus standard deviation(s); median value; fold changes. The multivariate discriminant analysis and other risk assessments can be performed on the free or commercially available computer statistical packages (SAS, SPSS, Matlab, R, etc.) or other statistical software packages or screening software known to those skilled in the art.

As obvious to one skilled in the art, in any of the embodiments discussed above, changing the risk cut-off level could change the results of the discriminant analysis for each subject. Statistics enables evaluation of the significance of each level. Commonly used statistical tests applied to a data set include t-test, f-test or even more advanced tests and methods of comparing data. Using such a test or method enables the determination of whether two or more samples are significantly different or not.

The significance may be determined by the standard statistical methodology known by the person skilled in the art. The chosen reference level may be changed depending on the mammal/subject for which the test is applied. Preferably, the subject according to the invention is a human subject, such as a subject considered at risk of having or developing stroke.

The chosen reference level may be changed if desired to give a different specificity or sensitivity as known in the art. Sensitivity and specificity are widely used statistics to describe and quantify how good and reliable a biomarker or a diagnostic test is. Sensitivity evaluates how good a biomarker or a diagnostic test is at detecting a disease, while specificity estimates how likely an individual (i.e. control, patient without disease) can be correctly identified as not sick.

Several terms are used along with the description of sensitivity and specificity; true positives (TP), true negatives (TN), false negatives (FN) and false positives. If a disease is proven to be present in a sick patient, and the diagnostic test confirms the presence of disease, the result of the diagnostic test is considered to be TP. If a disease is not present in an individual (i.e. control, patient without disease), and the diagnostic test confirms the absence of disease, the test result is TN. If the diagnostic test indicates the presence of disease in an individual with no such disease, the test result is FP. Finally, if the diagnostic test indicates no presence of disease in a patient with disease, the test result is FN.

### Sensitivity

Sensitivity=TP/(TP+FN)=number of true positive assessments/number of all samples from patients with disease. As used herein, the sensitivity refers to the measures of the proportion of actual positives, which are correctly identified as such—in analogy with a diagnostic test, i.e. the percentage of people having PaO2 below normal who are identified as having PaO2 below normal.

### Specificity

Specificity=TN/(TN+FP)=number of true negative assessments/number of all samples from controls. As used herein, the specificity refers to measures of the proportion of negatives, which are correctly identified. The relationship between both sensitivity and specificity can be assessed by the ROC curve. This graphical representation helps to decide the optimal model through determining the best threshold—or cut-off for a diagnostic test or a biomarker candidate.

As will be generally understood by those skilled in the art, methods for screening are processes of decision-making and therefore the chosen specificity and sensitivity depend on what is considered to be the optimal outcome by a given assay.

It would be obvious for a person skilled in the art that it may be advantageous to select a higher sensitivity at the expense of lower specificity in most cases, to identify as many patients with disease risk as possible.

In a preferred embodiment, the invention relates to a method with a high specificity, such as at least 70%, such as at least 80%, such as at least 90%, such as at least 95%, such as 100%. In another preferred embodiment, the invention relates to a method with a high sensitivity, such as at least 80%, such as at least 90%, such as 100%.

### EXAMPLES

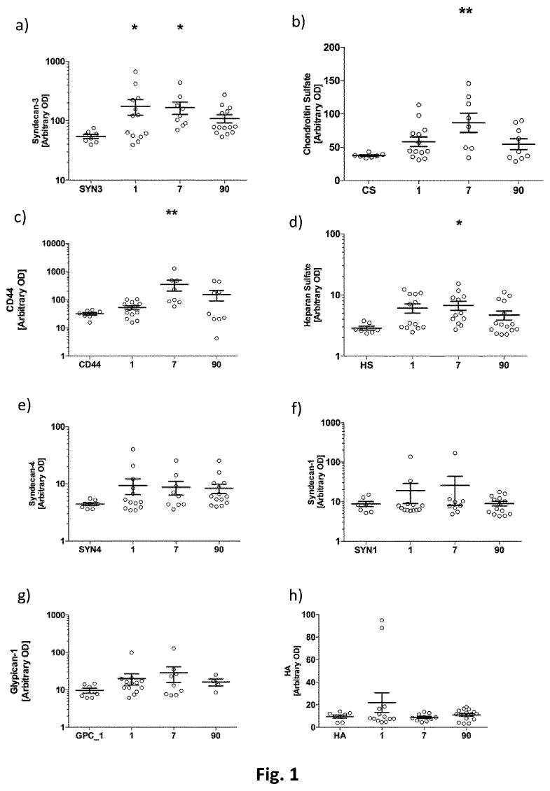

### Example 1: Blood from Human Stroke Patients (FIG. 1)

To show that biomarkers of the GLX structure are detectable in blood samples and that these biomarkers increase in response to disease and inflammation, blood samples obtained from patients day 1 (within 72 hours), 10 days, and 90 days after an acute stroke incident were tested and compared to the level of the GLX markers in blood samples from healthy individuals (the reference sample is indicated with the abbreviations for the GLX marker on the x-axis in the FIG. 1 a) to h)).

### Materials and Methods

### Plasma Dot Blotting

Antibody-based dot blots were used to assess GLX markers longitudinally. Two μl of plasma was dotted in duplicate on a cationic nitrocellulose membrane (Hybond N+, Amersham, GE Healthcare, Brondby, Denmark) and allowed to dry. The membrane was incubated for 60 minutes at room temperature in blocking buffer: 5% skim milk powder (Sigma-Aldrich) in tris-buffered saline+0.05% Tween20 (TBS-T; Sigma-Aldrich). The membrane was incubated thereafter with primary antibodies at their respective dilutions overnight, at 4° C. Membranes were thereafter washed in TBS-T and incubated with secondary antibodies conjugated to horseradish peroxidase, diluted at respective dilutions in blocking buffer, and raised against the source of the primary for 60 mins. Membranes were washed thoroughly with TBS-T and finally, in TBS. Membranes were visualized with Supersignal West femto luminescent substrate and Chemidoc XRS CCD camera (Bio-Rad Laboratories). Chemiluminescence was quantified with densitometry after normalizing to background with ImageJ software.

Primary antibodies: Heparan sulfate (HS) (1:1500, Millipore), Syndecan-1 (R&D Systems), Syndecan-4 (Santa Cruz)). Chondroitin sulfate (CS) (1:1000, Sigma-Aldrich), Syndecan-3 (1:1000, R&D Systems). HA (Bio-Rad), glypican-1 (R&D Systems), CD44 (1:200, DAKO/Aglient).

Secondary antibodies: HRP-conjugated: anti-rabbit (1:2000), anti-rat (1:4000), anti-mouse (1:3000) (DAKO/Agilent).

Visualisation and optical density analysis is performed as above with a secondary antibody-HRP complex and chemiluminescence (femtogram resolution). Tests were performed for the following biomarkers:

### Proteoglycans:

Syndecan-1 (FIG. 1 f))
Syndecan-3 (FIG. 1 a))
Syndecan-4 (FIG. 1 e))
Glypican-1 (FIG. 1g)) CD44 (FIG. 1 c))

### Glycosaminoglycans:

Chondroitin Sulfates (CS) (FIG. 1b))
Heparan Sulfates (HS) (FIG. 1 d))
Hyaluronic Acids (HA) (FIG. 1 h))

### Data Analysis:

Data sets were tested for normality (Shapiro-Wilk) and equal variance before statistical analyses were performed (One-way ANOVA). Data that was non-normal was log-transformed and re-tested for normality and equal variance. Data sets were then analyzed with parametric or non-parametric statistics based on normality after transformation. A P-value of <0.05 was reported as statistically significantly different. Data are presented as mean±S.E.M for normal data and median±interquartile range for non-normal data.

### Results

As seen in FIG. 1 a) to h), all GLX markers are detectable above background in all patient and healthy control samples. All GLX markers display a unique signature both between patients and healthy controls and within each patient over the timescale. As seen in FIG. 1, Syndecan-3, Chondroitin Sulfate (CS), CD44 and Heparan Sulfate (HS) are particularly effective in separating healthy control samples from Stroke patient sample at the 7-day time interval.

### Conclusion

In this set of experiments, it is shown that GLX components across different classes, are elevated and variable between Stroke patients and over time within each patient and thus should be considered as potential biomarkers of disease, disease severity, and treatment response. The classes of GLX that have shown to be of substantial relevance are glycosaminoglycans (GAGs) and proteoglycans (PGs).

When taken together, in here is provided evidence for the identification of GLX components consisting of: GAGs: Chondroitin Sulfates, Hyaluronic Acids, and Heparan Sulfates and PGs: Syndecans, and Glypicans; in biological samples. These represent the vast majority of the GLX structure and are all identified as being indicative of being biomarkers for Stroke disease.

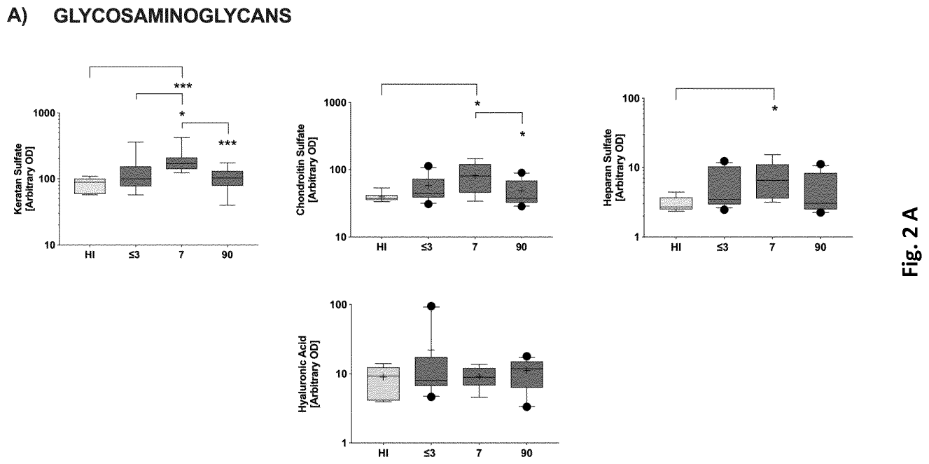

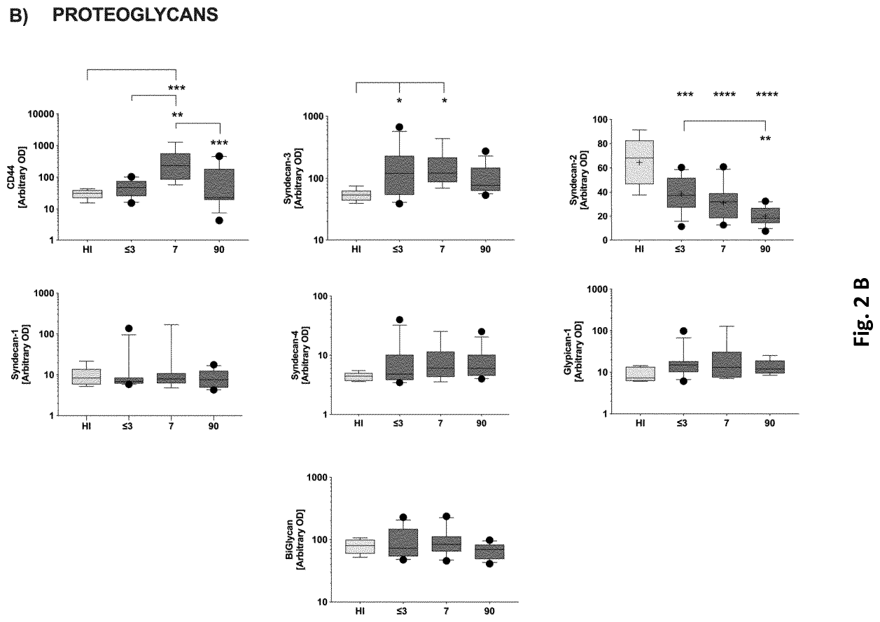

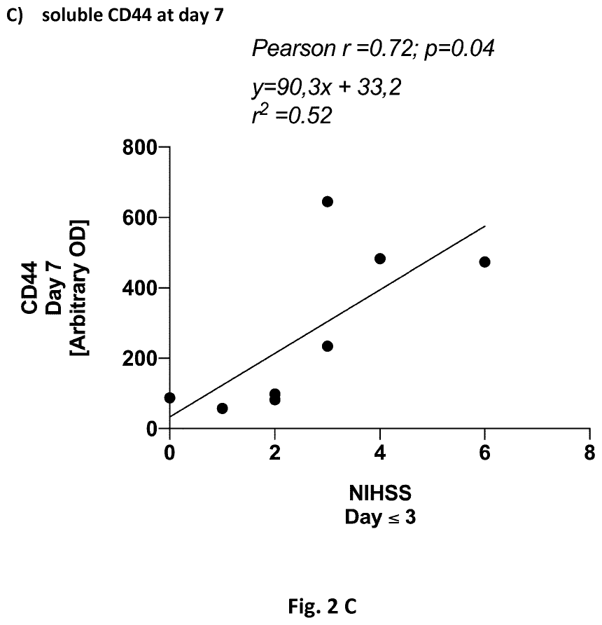

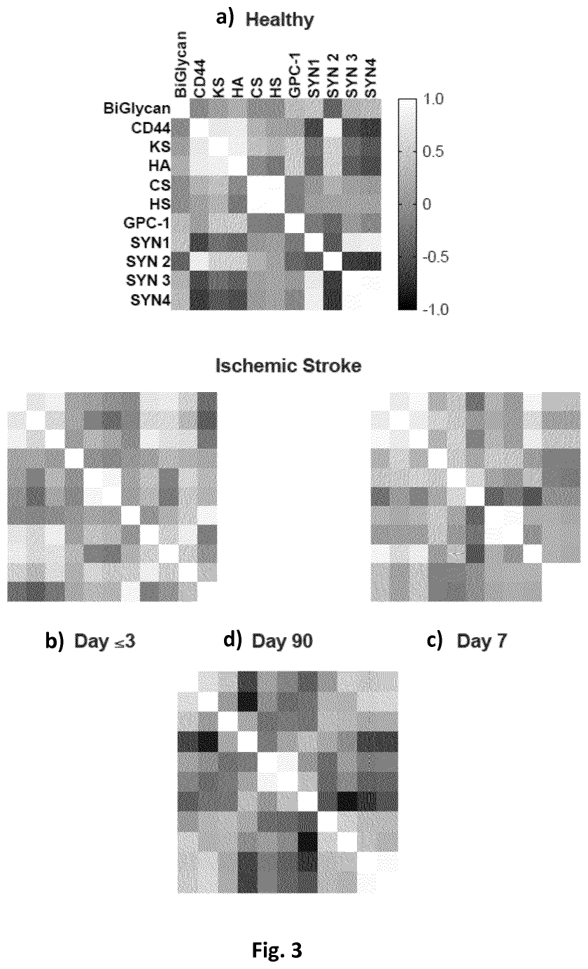

### Example 2: Blood from Human Stroke Patients (FIG. 2 and FIG. 3)

To show that biomarkers of the GLX structure are detectable in blood samples and that these biomarkers increase or decrease in response to disease and inflammation, blood samples obtained from patients day ≤3, 7 days, and 90 days after an acute stroke incident were tested and compared to the level of the GLX markers in blood samples from healthy age-matched individuals (HI). FIG. 2: GLX markers in the blood change as a result of inflammatory disease. GLX markers were measured in human plasma and presented as box and whisker plots (10-90 percentile, median line and ‘+’ for mean) in healthy individuals (HI) and ischemic stroke patients at day ≤3 (sample taken within 72 hours), Day 7, and Day 90 after stroke event. Data was labelled as significant with *, **, *** when p<0.05, 0.01, or <0.001, respectively. GLX markers in the blood change as a result of inflammatory disease.

FIG. 3: GLX markers in the blood change as a result of inflammatory disease. Correlation heat map of glycocalyx (GLX) markers in a) healthy controls and after ischemic stroke at b) Day 1 (within 72 hours after stroke), c) Day 7, and d) Day 90. GLX markers were measured in human plasma and Pearson correlations were mapped on a scale from −1 to 1. Values below 0 were plotted in red and values above zero in blue. GLX markers in the blood change as a result of inflammatory disease and also return towards baseline as stroke damage is resolved (Day 90), representing a soluble profile.

### Materials and Methods

### Patients

Plasma was stored at −20° C. after informed consent from patients with ISS at day ≤3 (within 72 hours N=13), day 7 (N=9), and day 90 (N=13) at the acute stroke unit, and from healthy individuals (HI, N=8). Patents were treated according to international stroke guidelines and received antithrombotic statin and, if appropriate, antihypertensive treatment. Exclusion criteria: transient cerebral ischemia, inflammatory diseases and cancer; cytostatic/immunosuppressive therapy; and cerebral, heart, eye or peripheral infarcts within 3 months. Neurological impairment was estimated using the National Institutes of Health Stroke Scale (NIHSS).

### Plasma GLX Analysis

Two microliters of plasma was dotted in duplicate on nylon membranes (Hybond N, Amersham, RPN203B). Membranes were blocked in blocking buffer (5% skim milk+TBS-0.05% Tween20) and incubated overnight at 4° C. in blocking buffer+primary antibody, washed (TBS-0.05% Tween20), incubated with blocking buffer+secondary antibody, washed, incubated with SuperSignal Femto Reagent (Thermo Scientific), imaged with CCD camera (LAS 4000, GE), analyzed with ImageJ for raw integrated density, and transformed by a factor of 10-5. Primary antibody targets: glycosaminoglycans (GAGs): chondroitin sulfate (CS, Sigma), heparin sulfate (HS, Millipore), keratin sulfate (KS, US-Biologicals), and hyaluronic acid (HA, Bio-Rad); and proteoglycans (PGs): CD44 (DAKO), syndecan (Syn)-1 (R&D), -2 (R&D), -3 (R&D), -4 (Santa Cruz), glypican-1 (R&D), and BiGlycan (Abcam). Secondary antibody targets: anti-mouse, -goat, -rat, -rabbit-HRP (DAKO).

### Data Analysis

Data sets were tested for normality (Shapiro-Wilk). Non-normal data sets were log-transformed and tested again. Data sets that remained non-normal were tested with nonparametric statistics. Parametric data were tested with OneWay ANOVA and nonparametric with Kruskal-Wallis test. Day 7 appeared to be the most dynamic sampling time. Therefore, we tested whether NISS at day ≤3 were predictive of day 7 levels. Data sets were tested with least squares regression modeling with ROUT (robust regression and outlier removal) correction (Q=1%) to approach a robust line of best fit. Residuals were tested for normality (QQ-Plot). Correlations were tested for normality, and Pearson coefficient was reported. Significant differences are reported when P<0.05 (GraphPad Prism 8).

Correlational heatmaps were generated from GLX measurements and Pearson correlations between the markers were mapped on a scale from −1 to 1. Values below 0 were plotted in red and values above zero in blue.

### A) Glycosaminoglycans:

Keratan Sulfate (KS)
Chondroitin Sulfates (CS)
Heparan Sulfate (HS)
Hyaluronic Acids (HA)

### B) Proteoglycans:

CD44
Syndecan-1
Syndecan-2
Syndecan-3
Syndecan-4
Glypican-1
BiGlycan

### Results

As seen in FIGS. 2 A) and B), all GLX markers are detectable above background in all patient and healthy control samples. All GLX markers display a unique signature both between patients and healthy controls and within each patient over the timescale. Five (KS, CS, HS, CD44 and Syndecan-3) of 11 GLX markers were significantly increased after ISS, and one was decreased (Syndecan-2). One-Way ANOVA tested differences in normal data and Kruskal-Wallis tested data that was non-normal after log-transformation. *, ***, and **** represent P values of <0.05, 0.01, 0.001 and 0.0001, respectively. Data presentation: median (line)/box (25^{th}-75^{th} percentile) and whiskers (10^{th}-90^{th} percentile); remaining data points as dots; and mean (+) for parametric raw data.

Keratan Sulfate, Chondroitin Sulfate, Heparan Sulfate, CD44, Syndecan-3 and Syndecan-2 are particularly effective in separating healthy control samples from Stroke patient sample at the 7 day time interval.

Keratan Sulfate and Syndecan-2 are particularly effective in separating healthy control samples from Stroke patient sample at the 90 day time interval.

As seen in FIG. 2C) soluble CD44 at day 7 significantly correlates to initial neurological impairment after minor stroke. Neurological impairment was estimated with the NIHSS within 72 h after ischemic stroke event (day ≤3). Plasma CD44 at day 7 correlated significantly to NIHSS (normal data, Person's r: 0.72, P<0.05; robust linear regression fit: 0.52).

As can be seen in FIG. 3 the relationships between each set of biomarkers has been analysed for the healthy control group and samples of stroke patients 1 day, 7 days and 90 days after stroke. The heatmeap correlational profile at day ≤3 after stroke clearly indicates an increased positive relationship between the levels of CD44 in combination with either BiGlycan, Syn1 or Syn3. Also, the heatmeap 1 day after stroke clearly indicates an increase of the levels of KS in combination with either BiGlycan, Syn1 or Syn3, an increase of the level of GPC-1 in combination with Syn3, and an increase of the levels of Syn-3 in combination with BiGlycan, CD44, KS, HA and Syn1. Other combinations with elevated or lowered levels of a combination of biomarkers 1 day after stroke can be found in FIG. 3.

The heatmeap correlation profile 7 days after stroke clearly indicates an increase of the levels of CD44 in combination with either BiGlycan, Syn1, Syn3 or Syn4. Also, the heatmeap 7 days after stroke clearly indicates an increase positive relationship between levels of KS in combination with either BiGlycan, Syn1, Syn3 or Syn4, an increase of the levels of GPC-1 in combination with either Syn1 or Syn2, and an increase of the levels of Syn-3 in combination with CD44, KS Syn-1 and Syn-4. Other combinations with elevated or lowered levels of a combination of biomarkers 7 days after stroke which are not described above are disclosed in FIG. 3.

The heatmap 90 days after stroke shows only a decreased number of correlating biomarkers with a biomarker profile returning to a similar but not yet identical to the profile of the heatmap of the healthy control group.

### Conclusion

In this set of experiments, it is shown that GLX components across different classes, are elevated and variable between stroke patients and over time within each patient and thus should be considered as potential biomarkers of disease, disease severity, and treatment response. The GLX components that have shown to be of substantial relevance are Keratan Sulfate, CD44, Syndecan-3 and Syndecan-2. In addition, GLX profiles (heatmaps) after stroke and over time from each other and from the healthy controls indicating the blood GLX profile can differentiate between disease and remission from disease.

We further conclude that there are one or more sets of two biomarkers which are elevated on day 1 and/or day 7 after stroke, indicating that there are certain relationships between some of the biomarkers as depicted in FIG. 3.

When taken together, in here is provided evidence for the identification of GLX components Keratan Sulfate, Chondroitin Sulfate, Heparan Sulfate, CD44, Syndecan-3 and Syndecan-2 in biological samples from stroke patients. These represent the vast majority of the GLX structure and are all identified as being indicative of being biomarkers for Stroke disease.

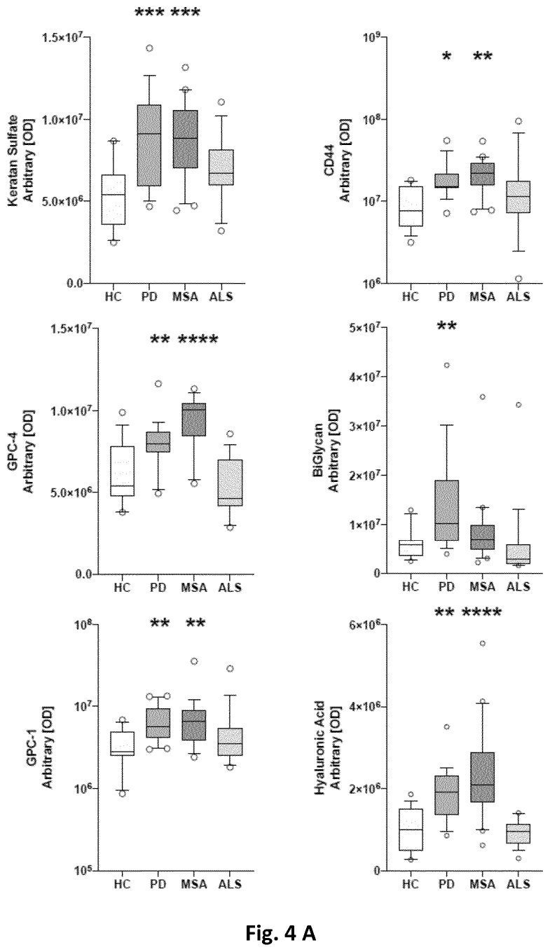

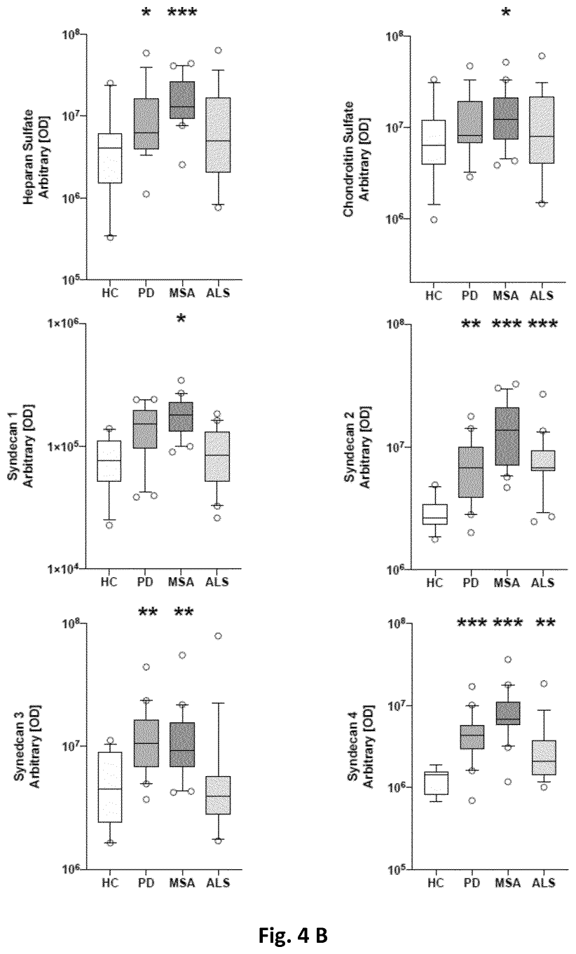

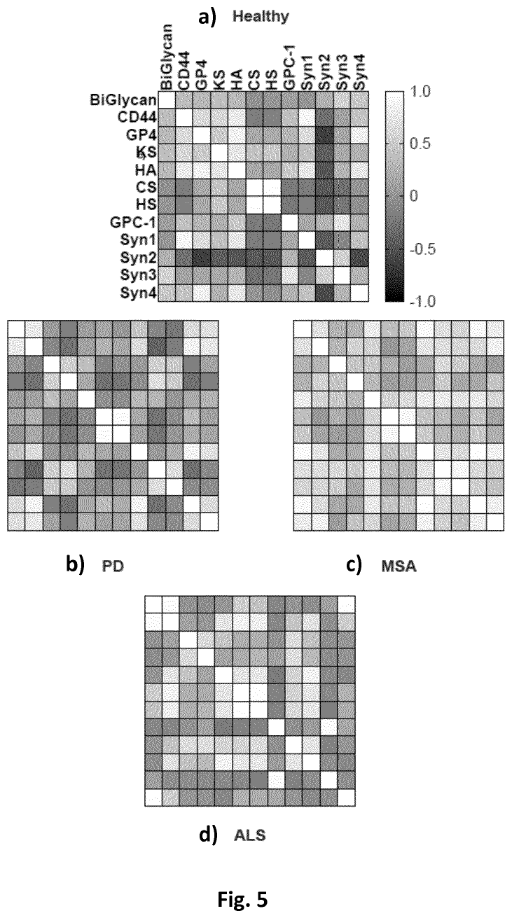

### Example 3: Blood from Patients with Neurodegenerative Disorders (FIG. 4 and FIG. 5)

To show that biomarkers of the GLX structure are detectable in blood samples and that the concentration of these biomarkers changes in response to disease and inflammation, blood samples obtained from patients with Parkinson's Disease (PD), Multiple System Atrophy (MSA) and Amyotrophic Lateral Sclerosis (ALS) were tested and compared to the level of the GLX markers in blood samples from healthy age-matched controls (HC) (y-axis: arbitrary OD). FIG. 4: GLX markers in the blood change as a result of inflammatory disease. GLX markers were measured in human plasma and presented as box and whisker plots (10-90 percentile, median line and ‘+’ for mean) in age/gender-matched healthy controls (HC) and Parkinson's Disease (PD), Multiple System Atrophy (MSA), and Amyotrophic Lateral Sclerosis (ALS). Data was labelled as significant with *, **, *** when p<0.05, 0.01, or <0.001, respectively. GLX markers in the blood change as a result of inflammatory disease.

FIG. 5: GLX markers in the blood change as a result of inflammatory disease. Correlation heat map of glycocalyx (GLX) markers in a) age/gender-matched healthy controls and b) Parkinson's Disease (PD), c) Multiple System Atrophy (MSA), d) Amyotrophic Lateral Sclerosis (ALS). GLX markers were measured in human plasma and Pearson correlations were mapped on a scale from −1 to 1. Values below 0 were plotted in red and values above zero in blue. GLX markers in the blood change as a result of inflammatory disease representing a soluble GLX profile.

### Materials and Methods

### Patients

Plasma samples were obtained from patients with PD, MSA, ALS and matched healthy controls (HC), N=20.

### Plasma GLX Analysis

Two microliters of plasma was dotted in duplicate on nylon membranes (Hybond N, Amersham, RPN203B). Membranes were blocked in blocking buffer (5% skim milk+TBS-0.05% Tween20) and incubated for one hour at room temperature blocking buffer+primary antibody, washed (TBS-0.05% Tween20), incubated with blocking buffer+secondary antibody, washed, incubated with SuperSignal Femto Reagent (Thermo Scientific), imaged with CCD camera (LAS 4000, GE), analyzed with ImageJ for raw integrated density. Primary antibody targets: glycosaminoglycans (GAGs): chondroitin sulfate (CS, Sigma), heparin sulfate (HS, Millipore), keratin sulfate (KS, US-Biologicals), and hyaluronic acid (HA, Bio-Rad); and proteoglycans (PGs): CD44 (DAKO), syndecan (Syn)-1 (R&D), -2 (R&D), -3 (R&D), -4 (Santa Cruz), glypican-1 (R&D), glypican-4 (Us Biologicals) and BiGlycan (Abcam). Secondary antibody targets: anti-mouse, -goat, -rabbit-HRP (DAKO) and rat-HRP (Sigma).

### Data Analysis

Data sets were tested for normality (Shapiro-Wilk). Non-normal data sets were log-transformed and tested again. Data sets that remained non-normal were tested with nonparametric statistics. Parametric data were tested with OneWay ANOVA and nonparametric with Kruskal-Wallis test. Significant differences are reported when P<0.05 (GraphPad Prism 8). *, **, ***, and **** represent P values of <0.05, 0.01, 0.001 and 0.0001, respectively.

Correlational heatmaps were generated from GLX measurements and Pearson correlations between the markers were mapped on a scale from −1 to 1. Values below 0 were plotted in red and values above zero in blue.

### GLX Structures:

### Keratan Sulfate (KS)

CD44
GPC-4
BiGlycan
Hyaluronic Acids (HA)
Chondroitin Sulfates (CS)
Heparan Sulfate (HS)
GPC-1
Syndecan-1
Syndecan-2
Syndecan-3
Syndecan-4

### Results

As seen in FIG. 4, all GLX markers are detectable above background in all patient and healthy control samples. All GLX markers display a unique signature both between patients and healthy controls.

For PD patients 10 of 12 GLX markers are significantly increased compared to the healthy control group, namely KS, CD44, GP4, BiGlycan, HA, HS, GPC-1, Syn2, Syn3 and Syn4. The strongest increase is observed for the biomarkers KS, HA, and Syn4.

For MSA patients 10 of 12 GLX markers are significantly increased compared to the healthy control group, namely KS, CD44, GP4, HA, Chondroitin Sulfate, HS, GPC-1, Syn2, Syn3, and Syn4. The strongest increase is observed for the biomarkers KS, CD44, GP4, HA, HS, Syn2, Syn3, and Syn4.

For ALS patients 2 of 12 GLX markers are significantly increased compared to the healthy control group, namely Syn2 and Syn4. The strongest increase is observed for the biomarker Syn2.

Biomarkers which are significantly increased in samples of all 3 diseases (PD, MSA and ALS) are Syn2 and Syn4. Biomarkers which are increased for two of the three diseases, namely PD and MSA, are KS, CD44, GP4, HA, HS, GPC-1, and Syn3.

As seen in FIG. 5 the relationships within each set of two biomarkers has been analysed for the healthy control group and samples of patients of Parkinson's Disease, MSA and ALS.

The heatmeap profiling of FIG. 5b) for patients with PD clearly indicates an increased positive relationship between levels of Syn3 and BiGlycan, CD44 and BiGlycan, Syn4 and BiGlycan, Syn4 and GPC4, CD44 and Syn3, CD44 and Syn4, Syn4 and GPC1, as well as for CD44 and GPC1.

The heatmeap of FIG. 5c) for patients with MSA clearly indicates an overall increase of the analysed biomarkers. Sets of biomarkers which are particularly positively increased are the sets of HA and BiGlycan, BiGlycan and GPC-1, BiGlycan and Syn3, CD44 and GPC-1, CD44 and Syn4, GP4 and Syn1, KS and Syn4, HA and GPC-1, HA and Syn1, HA and Syn2, HA and Syn3, CS and Syn3, CS and Syn1, CS and Syn2, CS and GPC1, HS and CD44, HS and GPC1, HS and Syn1, HS and Syn2, HS and Syn3, GPC-1 and GPC-1 and Syn3, GPC-1 and Syn4, as well as Syn3 and Syn4.

The heatmeap correlational profile of FIG. 5 d) for patients with ALS clearly indicates an increased relationship of the levels for the sets of BiGlycan and CD44, BiGlycan and HA, BiGlycan and CS, BiGlycan and HS, BiGlycan and Syn2, CD44 and CS, CD44 and HS, CD44 and Syn2, HA and Syn2, CS and Syn1, CS and Syn2, HS and Syn2, GPC1 and Syn4, as well as for Syn3 and Syn4.

Other combinations with elevated or lowered levels of a combination of biomarkers for PD, MSA and ALS which are not described above are disclosed in FIG. 5.

As seen in FIG. 5 a)-d) the heatmaps for PD, MSA and ALS show a certain pattern for each disease.

### Conclusion

In this set of experiments, it is shown that GLX components across different classes are elevated and variable for patients of PD, MSA and ALS and thus should be considered as potential biomarkers of disease. The GLX components that have shown to be of substantial relevance for patients of PD are Keratan Sulfate, CD44, BiGlycan, GPC-4, GPC-1, HA, Syn2, Syn3 and Syn4. The GLX components that have shown to be of substantial relevance for patients of MSA are Keratan Sulfate, CD44, GPC-4, GPC-1, HA, Syn2, Syn3 and Syn4. The GLX components that have shown to be of substantial relevance for patients of ALS are Syn2 and Syn4.

We further conclude that for each disease of PD, ALS and MSA there are one or more sets of two biomarkers which are particularly elevated, indicating that there are certain relationships between some of the biomarkers as depicted in FIG. 5. In addition, the typical pattern of correlation for each disease of PD, ALS and MSA can be used as a “fingerprint” to determine the condition of PD, ALS and/or MSA in a patient.

When taken together, in here is provided evidence for the identification of GLX components in biological samples from PD, MSA and/or ALS patients. Above listed GLX components are all identified as being indicative of being biomarkers for PD, MSA and/or ALS.

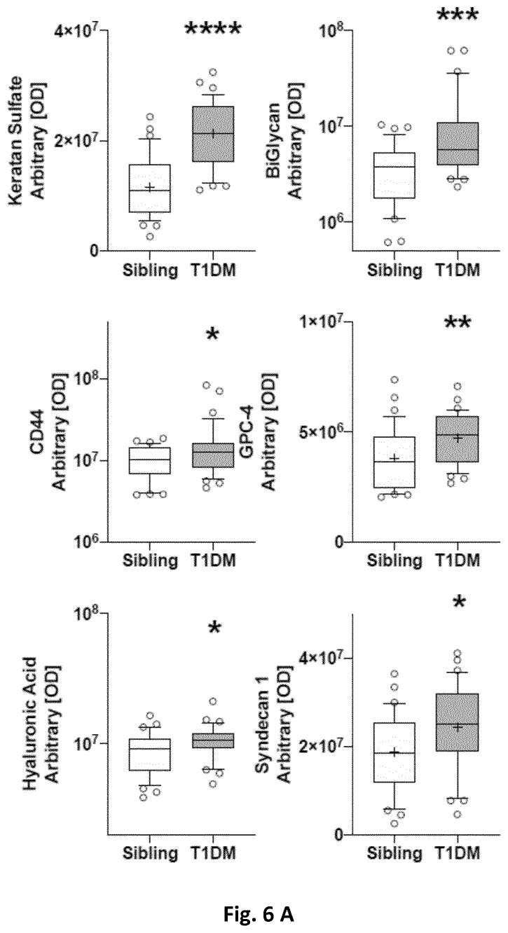

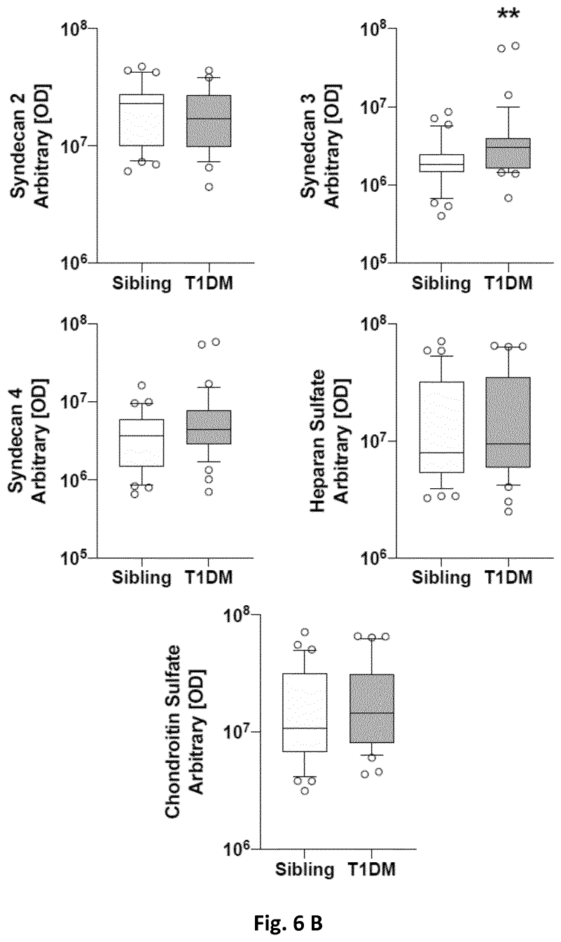

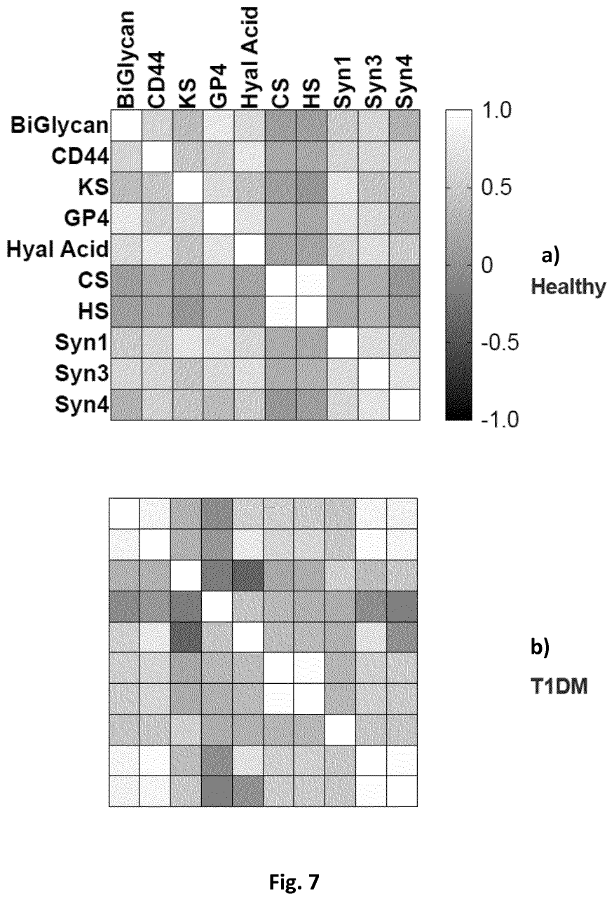

### Example 4: Blood from Patients with Autoimmune Disease, Type-I Diabetes Mellitus (FIG. 6 and FIG. 7)

To show that biomarkers of the GLX structure are detectable in blood samples and that the concentration of these biomarkers changes in response to disease and inflammation, blood samples obtained from children age 6-9 with autoimmune Type-I Diabetes Mellitus (T1DM) were tested and compared to the level of the GLX markers in blood samples from healthy sibling controls (Sibling) without T1DM (y-axis: arbitrary OD).

FIG. 6: GLX markers were measured in human plasma and presented as box and whisker plots (10-90 percentile, median line and ‘+’ for mean) in age/gender-matched healthy controls (siblings) and Type I Diabetes Mellitus (T1DM). Healthy controls are healthy siblings with a T1DM sibling in the family. Data was labelled as significant with *, **, *** when p<0.05, 0.01, or <0.001, respectively. GLX markers in the blood change as a result of inflammatory disease.

FIG. 7: GLX markers in the blood change as a result of inflammatory disease. Correlation heat map of glycocalyx (GLX) markers in a) age/gender-matched healthy controls and b) autoimmune disease Type I Diabetes mellitus (T1DM). GLX markers were measured in human plasma and Pearson correlations were mapped on a scale from −1 to 1. Values below 0 were plotted in red and values above zero in blue. GLX markers in the blood change as a result of inflammatory disease representing a soluble GLX profile.

### Materials and Methods

### Patients

Plasma was obtained from patients with autoimmune T1DM and matched controls from siblings whom are healthy but have a sibling with T1DM, N=33, 36, respectively.

### Plasma GLX Analysis

Two microliters of plasma was dotted in duplicate on nylon membranes (Hybond N, Amersham, RPN203B). Membranes were blocked in blocking buffer (5% skim milk+TBS-0.05% Tween20) and incubated at 4 C overnight in blocking buffer+primary antibody, washed (TBS-0.05% Tween20), incubated with blocking buffer+secondary antibody, washed, incubated with SuperSignal Femto Reagent (Thermo Scientific), imaged with CCD camera (LAS 4000, GE), analyzed with ImageJ for raw integrated density. Primary antibody targets: glycosaminoglycans (GAGs): chondroitin sulfate (CS, Sigma), heparin sulfate (HS, Millipore), keratin sulfate (KS, US-Biologicals), and hyaluronic acid (HA, Bio-Rad); and proteoglycans (PGs): CD44 (DAKO), syndecan (Syn)-1 (R&D), -2 (R&D), -3 (R&D), -4 (Santa Cruz), glypican-4 (Us Biologicals) and BiGlycan (Abcam). Secondary antibody targets: anti-mouse, -goat, -rabbit-HRP (DAKO) and rat-HRP (Sigma).

### Data Analysis

Data sets were tested for normality (Shapiro-Wilk). Non-normal data sets were log-transformed and tested again. Data sets that remained non-normal were tested with nonparametric statistics. Parametric data were tested with OneWay ANOVA and nonparametric with Kruskal-Wallis test. Significant differences are reported when P<0.05 (GraphPad Prism 8). *, **, ***, and **** represent P values of <0.05, 0.01, 0.001 and 0.0001, respectively.

Correlational heatmaps were generated from GLX measurements and Pearson correlations between the markers were mapped on a scale from −1 to 1. Values below 0 were plotted in red and values above zero in blue.

### GLX Structures:

Keratan Sulfate (KS)
CD44
GPC-4
BiGlycan
Chondroitin Sulfates (CS)
Heparan Sulfate (HS)
Hyaluronic Acid (HA)
Syndecan-1
Syndecan-2
Syndecan-3
Syndecan-4

### Results

As seen in FIG. 6, all GLX markers are detectable above background in all patient and healthy control samples. All GLX markers display a unique signature both between patients and healthy controls.

For T1DM patients 7 of 11 GLX markers are significantly increased compared to the healthy control group, namely KS, CD44, GP4, BiGlycan, HA, Syn1, and Syn3. The strongest increase is observed for the biomarkers KS, BiGlycan, GP4 and Syn3.

As seen in FIG. 7 the relationships within each set of two biomarkers has been analysed for the healthy sibling control group (FIG. 7a)) and samples of patients of T1DM (FIG. 7 b)).

The heatmeap for patients with T1DM clearly indicates an increase of the levels for the sets of BiGlycan and CD44, BiGlycan and Syn3, BiGlycan and Syn4, CD44 and Syn3, CD44 and Syn4, HA and BiGlycan, HA and Syn3, HA and Syn4, as well as for Syn3 and Syn4.

Other combinations with elevated or lowered levels of a combination of biomarkers for T1DM which are not described above are disclosed in FIG. 7.

### Conclusion

In this set of experiments, it is shown that GLX components across different classes are elevated and variable for patients of T1DM and thus should be considered as potential biomarkers of disease. The GLX components that have shown to be of substantial relevance for patients of T1DM are Keratan Sulfate, CD44, BiGlycan, GPC-4, Syn1 and Syn3.

We further conclude that for T1DM there are one or more sets of two biomarkers which are particularly elevated, indicating that there are certain relationships between some of the biomarkers as depicted in FIG. 7, that can differentiate between healthy and disease.

When taken together, in here is provided evidence for the identification of GLX components in biological samples from T1DM patients. The GLX components Keratan Sulfate, CD44, BiGlycan, GPC-4, Syn1 and Syn3 are all identified as being indicative of being biomarkers for T1DM.

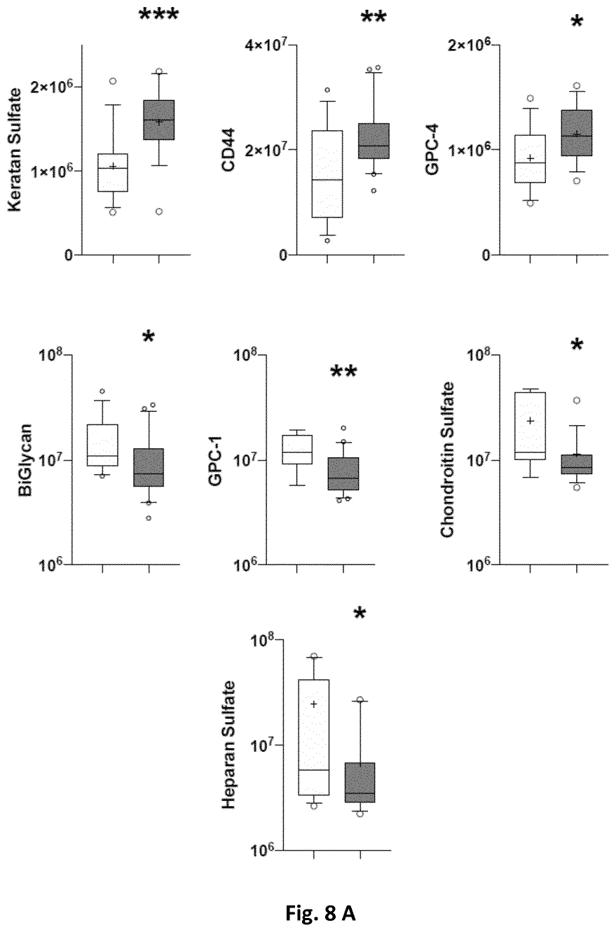

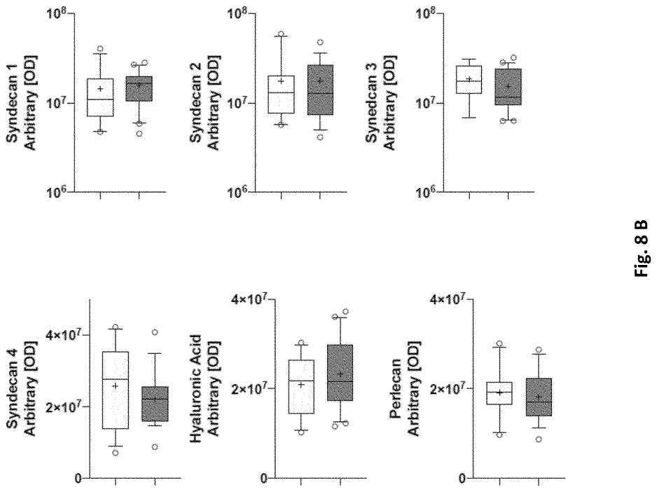

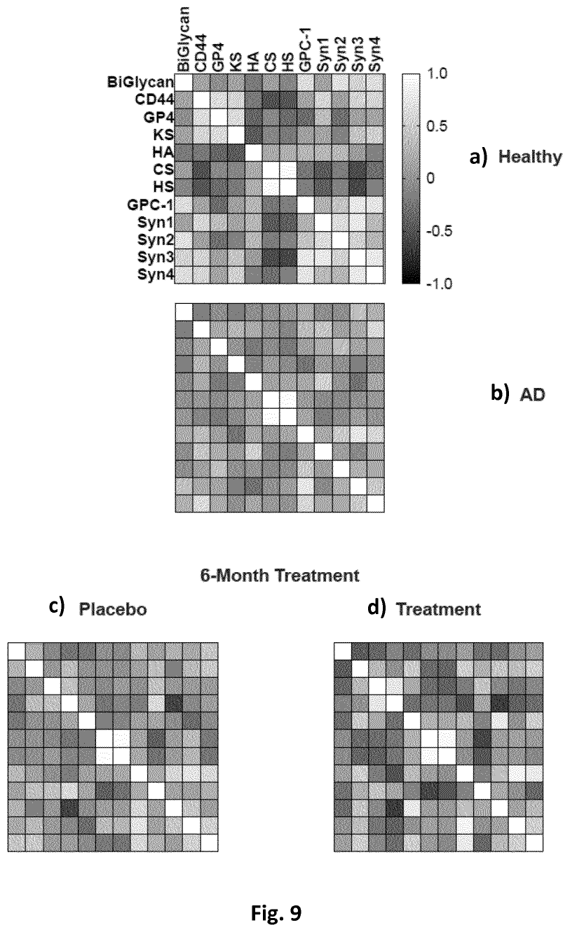

### Example 5: Blood from Patients with Alzheimer's Disease (FIG. 8 and FIG. 9)

To show that biomarkers of the GLX structure are detectable in blood samples and that the concentration of these biomarkers changes in response to disease and inflammation, blood samples obtained from patients with Alzheimer's Disease (AD, grey bars) were tested and compared to the level of the GLX markers in blood samples from healthy age-matched controls (HC, white bars) (y-axis: arbitrary OD).

FIG. 8: GLX markers in the blood change as a result of inflammatory disease. GLX markers were measured in human plasma and presented as box and whisker plots (10-90 percentile, median line and ‘+’ for mean) in age/gender-matched healthy controls (white) and Alzheimer's Disease (AD; Gray). Data was labelled as significant with *, **, *** when p<0.05, 0.01, or <0.001, respectively. GLX markers in the blood change as a result of inflammatory disease.

FIG. 9: GLX markers in the blood change as a result of inflammatory disease and respond to disease modifying drug treatment. Correlation heat map of glycocalyx (GLX) markers in a) age/gender-matched healthy controls and b) Alzheimer's Disease (AD) and AD patients after 6 months of treatment with c) placebo, and d) disease modifying drug treatment. GLX markers were measured in human plasma and Pearson correlations were mapped on a scale from −1 to 1. Values below 0 were plotted in red and values above zero in blue. GLX markers in the blood change as a result of inflammatory disease and treatment and represent a soluble GLX profile.

### Materials and Methods

### Patients

Plasma from AD patients (baseline (N=32)), healthy controls (N=20) were obtained and AD patients were followed up 6 months after receiving placebo (N=19) or disease treatment (N=13).

### Plasma GLX Analysis

Two microliters of plasma was dotted in duplicate on nylon membranes (Hybond N, Amersham, RPN203B). Membranes were blocked in blocking buffer (5% skim milk+TBS-0.05% Tween20) and incubated for one hour at room temperature in blocking buffer+primary antibody, washed (TBS-0.05% Tween20), incubated with blocking buffer+secondary antibody, washed, incubated with SuperSignal Femto Reagent (Thermo Scientific), imaged with CCD camera (LAS 4000, GE), analyzed with ImageJ for raw integrated density. Primary antibody targets: glycosaminoglycans (GAGs): chondroitin sulfate (CS, Sigma), heparin sulfate (HS, Millipore), keratin sulfate (KS, US-Biologicals), and hyaluronic acid (HA, Bio-Rad); and proteoglycans (PGs): CD44 (DAKO), syndecan (Syn)-1 (R&D), -2 (R&D), -3 (R&D), -4 (Santa Cruz), glypican-1 (R&D), glypican-4 (Us Biologicals), Perlecan (R&D), and BiGlycan (Abcam). Secondary antibody targets: anti-mouse, -goat, -rabbit-HRP (DAKO) and rat-HRP (Sigma).

### Data Analysis

Data sets were tested for normality (Shapiro-Wilk). Non-normal data sets were log-transformed and tested again. Data sets that remained non-normal were tested with nonparametric statistics. Parametric data were tested with OneWay ANOVA and nonparametric with Kruskal-Wallis test. Significant differences are reported when P<0.05. *, **, ***, and **** represent P values of <0.05, 0.01, 0.001 and 0.0001, respectively. (GraphPad Prism 8).

Correlational heatmaps were generated from GLX measurements and Pearson correlations between the markers were mapped on a scale from −1 to 1. Values below 0 were plotted in red and values above zero in blue.

### GLX Structures:

Keratan Sulfate (KS)
CD44
GPC-4
BiGlycan
Chondroitin Sulfates (CS)
Heparan Sulfate (HS)
Hyaluronic Acid
GPC-1
Syndecan-1
Syndecan-2
Syndecan-3
Syndecan-4
Perlecan

### Results

As seen in FIG. 8, all GLX markers are detectable above background in all patient and healthy control samples. All GLX markers display a unique signature both between patients and healthy controls.

For AD patients 7 of 13 GLX markers are significantly changed compared to the healthy control group, namely KS (increase), CD44 (increase), GP4 (increase), BiGlycan (decrease), GPC-1 (decreased), Chondroitin Sulfate (decreased), Heparan Sulfate (decreased). The strongest increase is observed for the biomarkers KS, CD44 and GPC-1.

As seen in FIG. 9 the heatmap of AD patients (FIG. 9b)) shows a different correlation between the biomarkers compared to the heatmap of the healthy control group (FIG. 9a)). Sets of biomarkers with increased positive relationships are e.g. the sets of CD44 and CS, CD44 and HS, GP4 and HS, GP4 and Syn2, KS and HA, KS and Syn2, BiGlycan and HA, HA and Syn4, CS and Syn3, CS and Syn4, as well as for HS and Syn3.

The biomarker heatmap for the 6-month placebo samples (FIG. 9c)) is similar but not identical to the heatmap of untreated AD patients (FIG. 9b)). In contrast, the biomarker heatmap for the samples of the 6-month treatment (FIG. 9d)) is comparably similar, but not identical, to the healthy control group and strongly dissimilar to the placebo samples.

Other combinations with elevated or lowered levels of a combination of biomarkers for AD which are not described above are disclosed in FIG. 9.

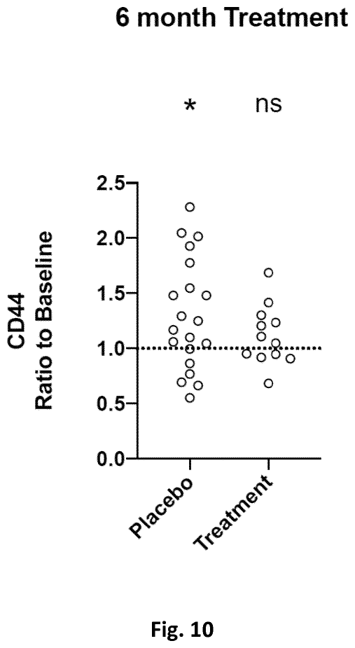

As seen in FIG. 10 the presence of CD44 in AD patients with a 6-month treatment is lowered compared to levels of CD44 in AD patients receiving placebo only.

### Conclusion

In this set of experiments, it is shown that GLX components across different classes are significantly increased or decreased, and thus different for patients of AD and thus should be considered as potential biomarkers of disease, disease severity, and treatment response. The GLX components that have shown to be of substantial relevance for patients of AD are Keratan Sulfate, CD44, GP4, BiGlycan, GPC-1 and Chondroitin Sulfate.

We further conclude that for AD there are one or more sets of two biomarkers which are particularly correlated, indicating that there are certain relationships between some of the biomarkers as depicted in FIG. 9. Furthermore, with the analysed biomarkers it is possible to monitor the progress of the treatment compared to a placebo only treatment. With this it is also possible to differentiate patients receiving a treatment from patients who do not receive a treatment at all or who only receive a poor treatment. Exemplary, the level of CD44 can differentiate between placebo receiving patients and treatment receiving patients after only 6 months.

When taken together, in here is provided evidence for the identification of GLX components in biological samples from AD patients. The GLX components KS, CD44, GP4, BiGlycan, GPC-1 and Chondroitin Sulfate are all identified as being indicative of being biomarkers for AD.

1. An ex vivo method of diagnosing and/or prognosticating at least one immune-mediated inflammatory disease (IMID) in a subject, said method comprising:
a. performing an in vitro measurement of the level of one or more biomarkers selected from the group consisting of glycosaminoglycans (GAGs) and proteoglycans (PGs) of the glycocalyx in a first biological sample from the subject;
b. optionally, performing an in vitro measurement of the level of the one or more biomarkers in a second or additional biological samples from the subject obtained at a later time point than said first biological sample;
c. comparing the level of said one or more biomarkers to the level of one or more corresponding references; and
d. using the measured value to evaluate the IMID state of the subject.

2-28. (canceled)

29. The ex vivo method according to claim 1, wherein the glycosaminoglycan (GAG) is keratan sulfate and/or the proteoglycan (PG) is selected from biGlycan, perlecan, glypican-1, glypican-4, syndecan-2, syndecan-3, syndecan-4, or CD44.

30. The ex vivo method according to claim 1, wherein the level of two or more biomarkers is measured.

31. The ex vivo method according to claim 1, wherein the state of the subject evaluated in step d. is determined as at risk of developing and/or having a given IMID, using said two or more biomarkers selected from keratan sulfate, biglycan, perlecan, glypican-1, glypican-4, syndecan-2 syndecan-3, syndecan-4 or CD44.

32. The ex vivo method according to claim 1, wherein the level of the one or more biomarkers is significantly greater or lower than the corresponding reference value with a P-value of at least <0.05.

33. The ex vivo method according to claim 1, wherein the biological sample is a blood sample, plasma, serum or cerebrospinal fluid.

34. The ex vivo method according to claim 1, wherein at least one additional biomarker is selected from one or more of chondroitin sulfate (CS), heparan sulfate (HS), hyaluronic acid (HA), and dermatan sulfate (DS), or keratan sulfate (KS).

35. The ex vivo method according to claim 1, wherein at least one additional biomarker is selected from one or more of syndecans, glypicans, or biglycan.

36. The ex vivo method according to claim 35, wherein at least one additional biomarker is selected from one or more of keratan sulfate, biglycan, perlecan, glypican-1, glypican-2, glypican-3, glypican-4, glypican-5, glypican-6, syndecan-2, syndecan-3, syndecan-4, or CD44

37. The ex vivo method according to claim 1, wherein the proteoglycan (PG) is selected from one or more of the group consisting of biglycan, CD44, perlecan, mimecan, decorin, hyalectans (aggrecan, versican, neurocan, brevican), betaglycan, lumincan, keratocan.

38. The ex vivo method according to claim 1, wherein the biomarkers are two or more biomarkers selected from the group consisting of biglycan, CD44, keratan sulfate, GPC-1, GPC-4, hyaluronic acid, chondroitin sulfate, heparan sulfate, perlecan, syndecan-1, syndecan-2, syndecan-3, and syndecan-4.

39. The ex vivo method according to claim 1, wherein the at least one IMID is an autoimmune disease, a neuroinflammatory disease, neurodegenerative disease, psychiatric disease, or an organ system disease.

40. The ex vivo method according to claim 1, wherein the at least one IMID is a stroke, Parkinson's Disease, Alzheimer's Disease, Multiple System Atrophy, Amyotrophic Lateral Sclerosis or Type-I Diabetes Mellitus.

41. The ex vivo method according to claim 1, wherein the amount of biological sample tested is higher than or equal to 0.01 μL.

42. The ex vivo method according to claim 1, wherein at least two biomarkers are measured simultaneously or consecutively.

43. The ex vivo method of claim 1 further comprising administering said subject a drug when the level of the biomarkers are above the corresponding one or more reference levels, wherein the drug administered for each respective IMID is selected from:

| Drug | IMID |
|---|---|
| Biologics | Autoimmune |
| Non-steroidal anti-inflammatory drugs | Disease |
| Glucocorticoids | |
| Disease-modifying antirheumatic drugs | |
| Biosimilars | |
| Vasodilators | Stroke |
| Statins | |
| Anticonvulsants | |
| ACE Inhibitors | |
| galantamine | Alzheimer's |
| donepezil | Disease |
| rivastigmine | |
| memantine | |
| citalopram | |
| mirtazapine | |
| sertraline | |
| bupropion | |
| duloxetine | |
| imipramine | |
| GLP-1 receptor agonists | |
| GIP receptor agonists | |
| SGLT-2 inhibitors | |
| Testosterone replacement | |
| memantine | |
| namzari | |
| Aducanumab | |
| Solanezumab | |
| Insulin | |
| verubecestat | |
| AADvac1 | |
| CSP-1103 | |
| intepiridine | |
| Levodopa | Parkinson's Disease |
| Carbidopa | |
| Carbidopa-levodopa | |
| Dopamine agonists | |
| MAO B inhibitors | |
| Catechol O-methyltransferase (COMT) inhibitors | |
| Anticholinergics | |
| Amantadine | |
| GLP-1 agonist | |
| GIP agonist | |
| SGLT-2 inhibitor | |

44. The method according to claim 43, wherein the GAG is Keratan Sulfate and/or wherein the PG is selected from BiGlycan, Glypican-1, Glypican-4, Syndecan-3 or CD44.

45. The method according to claim 43, wherein the drug and IMID, respectively, are selected from:

| Drug | IMID |
|---|---|
| Immunosuppressant Drugs | Lupus |
| Cyclophosphamide | |
| Azathioprine | |
| Hydroxychloroquine | |
| Methotrexate | |
| Ibuprofen | |
| Naproxen | |
| Sulindac | |
| misoprostol | |
| Prednisolone | |
| methylprednisolone | |
| butesonide | |
| chloroquine | |
| dapsone | |
| chlorambucil | |
| mycophenolate mofetil | |
| Belimumab | |
| Rituximab | |
| dehydroepiandrosterone | |
| Immunosuppressant Drugs | Rheumatoid Arthritis |
| Cyclophosphamide | |
| Azathioprine | |
| Belimumab | |
| Hydroxychloroquine | |
| Rituximab | |
| Ibuprofen | |
| Etanercept | |
| methotrexate | |
| sulfasalazine | |
| leflunomide | |
| Infliximab | |
| prednisolone | |
| methylprednisolone | |
| butesonide | |
| Immunosuppressant Drugs | Inflammatory Bowel Disease |
| Mesalazine | |
| prednisolone | |
| methylprednisolone | |
| butesonide | |
| Azathioprine | |
| methotrexate | |
| cyclosporin | |
| tacrolimus | |
| adalimumab | |
| Infliximab | |
| certolizumab pegol | |
| Ornidazole | |
| Metronidazole | |
| clarithromycin | |
| rifaximin ciprofloxacin | |
| infliximab | Celiac Disease |
| infliximab-abda | |
| infliximab-dyyb | |
| balsalazide | Crohn's Disease |
| Mesalamine | |
| olsalazine | |
| Sulfasalazine | |
| Tacrolimus | |
| prednisolone | |
| methylprednisolone | |
| Butesonide | |
| Azathioprine | |
| methotrexate | |
| Cyclosporin | |
| Golimumab | |
| adalimumab-adbm | |
| Adalimumab | |
| Infliximab | |
| infliximab-abda | |
| infliximab-dyyb | |
| vedolizumab | |
| balsalazide | Ulcerative Colitis |
| Mesalamine | |
| olsalazine | |
| Sulfasalazine | |
| prednisolone | |
| methylprednisolone | |
| Butesonide | |
| Azathioprine | |
| methotrexate | |
| cyclosporin | |
| golimumab | |
| adalimumab-adbm | |
| adalimumab | |
| Infliximab | |
| infliximab-abda | |
| infliximab-dyyb | |
| vedolizumab | |
| Calcipotriene | Psoriasis |
| Acitretin | |
| Methoxsalen | |
| Trioxsalen | |
| Adalimumab | |
| Etanercept | |
| Infliximab | |
| Secukinumab | |
| Ixekizumab | |
| Apremilast | |
| Methotrexate | |
| Cyclosporin | |
| Atorvastatin | Stroke |
| fluvastatin | |
| lovastatin | |
| Pitavastatin | |
| Pravastatin | |
| Rosuvastatin | |
| simvastatin | |
| Coumadin | |
| Jantoven | |
| Marfarin | |
| Clopidogrel | |
| Tissue plasminogen activator | |
| angiotensin-converting enzyme inhibitors | |
| beta-blockers | |
| calcium channel blockers | |
| GLP-1 agonist | |
| GIP agonist | |
| SGLT-2 inhibitor | |
| Galantamine | Alzheimer's Disease |
| Donepezil | |
| Rivastigmine | |
| Memantine | |
| Citalopram | |
| Mirtazapine | |
| Sertraline | |
| Bupropion | |
| Duloxetine | |
| Imipramine | |
| GLP-1 receptor agonists | |
| GIP receptor agonists | |
| SGLT-2 inhibitors | |
| Testosterone replacement | |

and/or selected from

| | |
|---|---|
| INN | Common brand names |

Psychiatric Disease
Anxiety Disorders

| | |
|---|---|
| Alprazolam | Xanax |
| Bromazepam | Lexotanil |
| Chlordiazepoxide | Librium |
| Clobazam | Frisium |
| Clonazepam | Klonopin |
| Clorazepate | Tranxene |
| Diazepam | Valium |
| Lorazepam | Ativan, Temesta |
| Oxazepam | Serax |
| Tofisopam | Emandaxin, Grandaxin |

Non-Benzodiazepine Anxiolytics

| | |
|---|---|
| Buspirone | BuSpar, Spitomin |
| Hydroxyzine | Atarax, Vistaril |
| Meprobamate | Equanil, Miltown |
| Gabapentin | Neurontin, Gabaran |
| Pregabalin | Lyrica |

Antidepressants

| | |
|---|---|
| Citalopram | Celexa, Cipramil |
| Clomipramine | Anafranil |
| Doxepin | Doxepin, Sinequan |
| Escitalopram | Cipralex, Lexapro |
| Fluoxetine | Prozac, Sarafem |
| Fluvoxamine | Fevarin, Luvox |
| Imipramine | Tofranil |
| Mirtazapine | Avanza, Remeron, Zispin |
| Paroxetine | Paxil, Pexeva, Seroxat |
| Sertraline | Lustral, Zoloft |
| Trazodone | Azona, Deprax, Desyrel, Oleptro, Trittico, |
| | Thombran |
| 5-HTP | |
| Tryptophan | |

Autism

| | |
|---|---|
| Aripiprazole | Abilify |
| Risperidone | Risperdal |

Bipolar Disorder
Mood Stabilizers

| | |
|---|---|
| Carbamazepine | Carbatrol, Carnevix, Epitol, Equetro, Tegretol, |
| | Tegretol XR, Teril |
| Gabapentin | Neurontin |
| Lamotrigine | Lamictal |
| Levetiracetam | Keppra |
| Lithium salts | Camcolit, Eskalith, Lithobid, Sedalit |
| Oxcarbazepine | Trileptal |
| Topiramate | Topamax |
| Sodium valproate | Convulex, Depakene, Depakine Enteric, Orfiril, |
| [note 1] | Stavzor |
| Divalproex | Depakote, Epival, Ergenyl Chrono |
| sodium [note 2] | |
| Sodium valproate | Depakine Chrono, Depakine Chronosphere, |
| and | |
| valproic acid in | Epilim Chrono, Epilim Chronosphere |
| 2.3:1 ratio | |

Atypical Antipsychotics

| | |
|---|---|
| Aripiprazole | Abilify |
| Asenapine | Saphris, Sycrest |
| Olanzapine | Zyprexa |
| Quetiapine | Seroquel |
| Risperidone | Risperdal |
| Ziprasidone | Geodon, Zeldox |

Depressive Disorders

| | |
|---|---|
| Amisulpride | Amazeo, Amipride, Amival, Deniban, Solian, |
| | Soltus, Sulpitac, Sulprix |
| Amitriptyline | Elavil, Endep, Tryptanol, Tryptomer |
| Agomelatine | Valdoxan, Melitor, Thymanax |
| Bupropion | Aplenzin, Wellbutrin |
| Citalopram | Celexa, Cipramil |
| Clomipramine | Anafranil |
| Desipramine | Norpramin, Pertofrane |
| Desvenlafaxine | Pristiq |
| Doxepin | Aponal, Adapine, Deptran, Prudoxin, Silenor, |
| | Sinquan, Sinequan, Zonalon |
| Duloxetine | Cymbalta |
| Escitalopram | Cipralex, Lexapro |
| Fluoxetine | Prozac, Sarafem |
| Fluvoxamine | Luvox, Faverin |
| Imipramine | Antideprin, Tofranil |
| Lamotrigine | Lamictal |
| Levomilnacipran | Fetzima |
| Mirtazapine | Remeron, Avanza |
| Moclobemide | Aurorix, Manerix |
| Nortriptyline | Aventyl, Pamelor |
| Paroxetine | Paxil, Pexeva, Seroxat |
| Phenelzine | Nardil |
| Protriptyline | Vivactil |
| Reboxetine | Edronax, Norebox, Prolift, Solvex, Vestra |
| Rubidium | Rubinorm |
| chloride | |
| Selegiline | Emsam |
| Sertraline | Zoloft, Lustral |
| Tianeptine | Stablon, Coaxil, Tatinol |
| Tranylcypromine | Parnate |
| Trazodone | Azona, Deprax, Desyrel, Oleptro, Trittico, |
| | Thombran |
| Venlafaxine | Effexor, Effexor XR |
| Vilazodone | Viibryd |
| Vortioxetine | Trintellix |
| Chloral hydrate | Chloraldurat, Somnote |
| Clomethiazole | Distraneurin, Heminevrin |
| Glutethimide | Doriden |
| Niaprazine | Nopron |
| Sodium oxybate | Alcover, Xyrem |
| Tizanidine | Sirdalud, Zanaflex |
| Amitriptyline | Elavil, Endep, Laroxyl, Lentizol, Saroten, Sarotex, |
| | Tryptizol, Tryptomer |
| Doxepin | Doxepin, Silenor |
| Mianserin | Bolvidon, Depnon, Lerivon, Tolvon |
| Mirtazapine | Avanza, Remeron, Zispin |
| Trazodone | Azona, Deprax, Desyrel, Oleptro, Trittico, |
| | Thombran |
| Trimipramine | Rhotrimine, Stangyl, Surmontil |

Psychotic Disorders

| | |
|---|---|
| Chlorprothixene | Truxal |
| Levomepromazine | Levium, Levomepromazine Neuraxph, Neurocil |
| Perazine | Perazin Neuraxph, Taxilan |
| Promethazine | Atosil, Closin, Promethazin Neuraxph, Proneurin, |
| | Prothazin |
| Prothipendyl | Dominal |
| Sulpiride | Dogmatil, Dogmatyl, Sulpirid |
| Thioridazine | Mellaril, Thioridazin Neuraxph |
| Zuclopenthixol | Cisordinol, Clopixol |
| Perphenazine | Trilafon |
| Benperidol | Benperidol Neuraxph, Glianimon |
| Bromperidol | Impromen |
| Fluphenazine | decanoate |
| enanthate | Dapotum Injektion, Flunanthate, Moditen |
| | Enanthate Injection, Sinqualone Enantat |
| hydrochloride | Dapotum, Permitil, Prolixin, Lyogen, Moditen, |
| | Omca, Sediten, Selecten, Sevinol, Siqualone, |
| | Trancin |
| Fluspirilen | Fluspi, Fluspirilen Beta, Imap |
| Haloperidol | Haldol, Serenase |
| Pimozide | Orap |
| Amisulpride | Solian |
| Aripiprazole | Abilify |
| Asenapine | Saphris |
| Clozapine | Clozaril, Fazaclo, Leponex |
| Iloperidone | Fanapt |
| Lurasidone | Latuda |
| Melperone | Eunerpan, Melneurin |
| Olanzapine | Zyprexa, Zyprexa Relprevv |
| Paliperidone | Invega, Invega Sustenna |
| Quetiapine | Seroquel |
| Risperidone | Risperdal, Risperdal Consta |
| Ziprasidone | Geodon, Zeldox |
| Zotepine | Nipolept |
| Carbamazepine | Tegretol |
| Lamotrigine | Lamictal |

46. A method of treating a stroke, Parkinson's Disease (PD), Multiple System Atrophy (MSA), Amyotrophic Lateral Sclerosis (ALS), Type-I Diabetes Mellitus (T1DM) or Alzheimer's Disease (AD) in an individual known to have at least one or more biomarkers selected from the group consisting of glycosaminoglycans (GAGs) and proteoglycans (PGs) of the glycocalyx comprising administering a drug selected from the following for the respective IMID

| Drug | IMID |
|---|---|
| Biologics | Autoimmune |
| Non-steroidal anti-inflammatory drugs | Disease |
| Glucocorticoids | |
| Disease-modifying antirheumatic drugs | |
| Biosimilars | |
| atorvastatin | Stroke |
| fluvastatin | |
| lovastatin | |
| pitavastatin | |
| pravastatin | |
| rosuvastatin | |
| simvastatin | |
| coumadin | |
| jantoven | |
| marfarin | |
| clopidogrel | |
| tissue plasminogen activator | |
| angiotensin-converting enzyme inhibitors | |
| beta-blockers | |
| calcium channel blockers | |
| GLP-1 agonist | |
| GIP agonist | |
| SGLT-2 inhibitor | |
| galantamine | Alzheimer's |
| donepezil | Disease |
| rivastigmine | |
| memantine | |
| citalopram | |
| mirtazapine | |
| sertraline | |
| bupropion | |
| duloxetine | |
| imipramine | |
| GLP-1 receptor agonists | |
| GIP receptro agonists | |
| SGLT-2 inhibitors | |
| Testosterone replacement | |
| memantine | |
| namzari | |
| Aducanumab | |
| Solanezumab | |
| Insulin | |
| verubecestat | |
| AADvac1 | |
| CSP-1103 | |
| intepiridine | |
| Levodopa | Parkinson's |
| Carbidopa | Disease |
| Carbidopa-levodopa | |
| Dopamine agonists | |
| MAO B inhibitors | |
| Catechol O-methyltransferase (COMT) | |
| inhibitors | |
| Anticholinergics | |
| Amantadine | |
| GLP-1 agonist | |
| GIP agonist | |
| SGLT-2 inhibitor | |

47. The method according to claim 46, wherein the glycosaminoglycan (GAG) is Keratan Sulfate and/or the proteoglycan (PG) is selected from BiGlycan, Glypican-1, Glypican-4, Syndecan-3 or CD44.

48. A kit comprising:
binding agents for at least two biomarkers selected from the group consisting of glycosaminoglycans (GAGs) and proteoglycans (PGs) of the glycocalyx; and
optionally instructions for using the binding agents in the evaluation of IMID.

49. The kit according to claim 48, wherein the glycosaminoglycan (GAG) is Keratan Sulfate and/or the proteoglycan (PG) is selected from BiGlycan, Glypican-1, Glypican-4, Syndecan-3 or CD44.

50. A kit comprising:
binding agents for Keratan Sulfate, BiGlycan, Glypican-1, Glypican-4, Syndecan-3 or CD44; and
instructions for using the binding agents in the evaluation of stroke, Parkinson's Disease (PD), Multiple System Atrophy (MSA), Amyotrophic Lateral Sclerosis (ALS), Type-I Diabetes Mellitus (T1DM) or Alzheimer's Disease (AD).

51. A microfluidic detection chip for the detection of an IMID in a patient said chip comprising one or more separation channels which are coated with one or more binding agents for the biomarkers selected from the group consisting of glycosaminoglycans (GAGs) and proteoglycans (PGs) of the glycocalyx.

52. The microfluidic detection chip according to claim 51, wherein the glycosaminoglycan (GAG) is selected from keratan sulfate; and/or the proteoglycan (PG) is selected from biglycan, glypican-1, glypican-4, syndecan-3, syndecan-4 or CD44.

53. The microfluidic detection chip according to claim 51, said chip comprising one or more layers in which a plurality of separation channels are disposed in each layer; at least one sample inlet port which is in fluid communication with the separation channels and into which a biological sample is introduced; wherein one or more of the separation channels have a plurality of three-dimensional (3D) separation zones; with an intermittent partition zone wherein the chip is an optical chip.

54. The kit according to claim 48, further comprising a microfluidic detection chip for the detection of an IMID in a patient said chip comprising one or more separation channels which are coated with one or more binding agents for the biomarkers selected from the group consisting of glycosaminoglycans (GAGs) and proteoglycans (PGs) of the glycocalyx.
